SEQUENCE LISTING (80 sequences)

SEQ ID NO: 1 - protein, 7 aa, synthetic construct
FPGSDRF

SEQ ID NO: 2 - protein, 9 aa, synthetic construct
RRRRRRRRR

SEQ ID NO: 3 - protein, 10 aa, synthetic construct
RRRRRRRRRF

SEQ ID NO: 4 - protein, 5 aa, synthetic construct
  REPEAT 2
AGRGD

SEQ ID NO: 5 - protein, 11 aa, synthetic construct
FPGSDRFGRGD

SEQ ID NO: 6 - protein, 14 aa, synthetic construct
FPGSDRFGGGGRGD

SEQ ID NO: 7 - protein, 20 aa, synthetic construct
FPGSDRFGGGGGGGGGGRGD

SEQ ID NO: 8 - protein, 11 aa, synthetic construct
FPGSDRFARGD

SEQ ID NO: 9 - protein, 12 aa, synthetic construct
FPGSDRFAGRGD

SEQ ID NO: 10 - protein, 15 aa, synthetic construct
FPGSDRFAGGGGRGD

SEQ ID NO: 11 - protein, 21 aa, synthetic construct
FPGSDRFAGGGGGGGGGGRGD

SEQ ID NO: 12 - protein, 11 aa, synthetic construct
FFPGSDRFRGD

SEQ ID NO: 13 - protein, 12 aa, synthetic construct
FFPGSDRFGRGD

SEQ ID NO: 14 - protein, 15 aa, synthetic construct
FFPGSDRFGGGGRGD

SEQ ID NO: 15 - protein, 21 aa, synthetic construct
FFPGSDRFGGGGGGGGGGRGD

SEQ ID NO: 16 - protein, 12 aa, synthetic construct
FFPGSDRFARGD

SEQ ID NO: 17 - protein, 13 aa, synthetic construct
FFPGSDRFAGRGD

SEQ ID NO: 18 - protein, 22 aa, synthetic construct
FFPGSDRFAGGGGGGGGGGRGD

SEQ ID NO: 19 - protein, 19 aa, synthetic construct
RRRRRRRRRFPGSDRFRGD

SEQ ID NO: 20 - protein, 29 aa, synthetic construct
RRRRRRRRRFPGSDRFGGGGGGGGGGRGD

SEQ ID NO: 21 - protein, 20 aa, synthetic construct
RRRRRRRRRFPGSDRFARGD

SEQ ID NO: 22 - protein, 30 aa, synthetic construct
RRRRRRRRRFPGSDRFAGGGGGGGGGGRGD

SEQ ID NO: 23 - protein, 20 aa, synthetic construct
RRRRRRRRRFFPGSDRFRGD

SEQ ID NO: 24 - protein, 30 aa, synthetic construct
RRRRRRRRRFFPGSDRFGGGGGGGGGGRGD

SEQ ID NO: 25 - protein, 21 aa, synthetic construct
RRRRRRRRRFFPGSDRFARGD

SEQ ID NO: 26 - protein, 31 aa, synthetic construct
RRRRRRRRRFFPGSDRFAGGGGGGGGGGRGD

SEQ ID NO: 27 - protein, 10 aa, synthetic construct
FPGSDRFRGD

SEQ ID NO: 28 - protein, 10 aa, synthetic construct
FPGSDRFRGD

SEQ ID NO: 29 - DNA, 22 bp, synthetic construct
  source 1..22: mol_type = other DNA
ccttggatggagaactactccg

SEQ ID NO: 30 - DNA, 22 bp, synthetic construct
  source 1..22: mol_type = other DNA
tccgtagtgacagccagaagct

SEQ ID NO: 31 - DNA, 22 bp, synthetic construct
  source 1..22: mol_type = other DNA
catccacagtgactgcatgctg

SEQ ID NO: 32 - DNA, 22 bp, synthetic construct
  source 1..22: mol_type = other DNA
aaggctggaacagaccaagtgg

SEQ ID NO: 33 - DNA, 23 bp, synthetic construct
  source 1..23: mol_type = other DNA
catcactgccacccagaagactg

SEQ ID NO: 34 - DNA, 23 bp, synthetic construct
  source 1..23: mol_type = other DNA
atgccagtgagcttcccgttcag

SEQ ID NO: 35 - DNA, 23 bp, synthetic construct
  source 1..23: mol_type = other DNA
gtggatgctcttcagttcgtgtg

SEQ ID NO: 36 - DNA, 22 bp, synthetic construct
  source 1..22: mol_type = other DNA
tccagtctcctcagatcacagc

SEQ ID NO: 37 - DNA, 22 bp, synthetic construct
  source 1..22: mol_type = other DNA
gtcctgaaggctcagacttggt

SEQ ID NO: 38 - DNA, 23 bp, synthetic construct
  source 1..23: mol_type = other DNA
ccagccgtaaatactgcaagtgg

SEQ ID NO: 39 - DNA, 23 bp, synthetic construct
  source 1..23: mol_type = other DNA
gttcgccttcattatggactgcc

SEQ ID NO: 40 - DNA, 22 bp, synthetic construct
  source 1..22: mol_type = other DNA
atagcaccctgttcccgcaaag

SEQ ID NO: 41 - DNA, 22 bp, synthetic construct
  source 1..22: mol_type = other DNA
actgtcaggcgacacttccatg

SEQ ID NO: 42 - DNA, 22 bp, synthetic construct
  source 1..22: mol_type = other DNA
gtgctcctgtttgacctgaggt

SEQ ID NO: 43 - DNA, 23 bp, synthetic construct
  source 1..23: mol_type = other DNA
cgagagtgcaaatggcaattccg

SEQ ID NO: 44 - DNA, 22 bp, synthetic construct
  source 1..22: mol_type = other DNA
gatgaacgctgtttctcggcag

SEQ ID NO: 45 - DNA, 22 bp, synthetic construct
  source 1..22: mol_type = other DNA
aactgcaccaccgtcagcaaca

SEQ ID NO: 46 - DNA, 22 bp, synthetic construct
  source 1..22: mol_type = other DNA
agcgtgtcactgcgaaagctac

SEQ ID NO: 47 - DNA, 22 bp, synthetic construct
  source 1..22: mol_type = other DNA
ttctcgtcgctttgtggatgcc

SEQ ID NO: 48 - DNA, 23 bp, synthetic construct
  source 1..23: mol_type = other DNA
caccgtgacacttacattccagc

SEQ ID NO: 49 - DNA, 22 bp, synthetic construct
  source 1..22: mol_type = other DNA
gctcctgttcttcctactgctg

SEQ ID NO: 50 - DNA, 22 bp, synthetic construct
  source 1..22: mol_type = other DNA
atgtcaggcacactgtgttggc

SEQ ID NO: 51 - DNA, 22 bp, synthetic construct
  source 1..22: mol_type = other DNA
accacgacatggacttcggaga

SEQ ID NO: 52 - DNA, 22 bp, synthetic construct
  source 1..22: mol_type = other DNA
ccgcttcaggttttccgttacc

SEQ ID NO: 53 - DNA, 23 bp, synthetic construct
  source 1..23: mol_type = other DNA
ctccagaccattaacctcagtgc

SEQ ID NO: 54 - DNA, 22 bp, synthetic construct
  source 1..22: mol_type = other DNA
caccaccagatccatgaagacc

SEQ ID NO: 55 - DNA, 21 bp, synthetic construct
  source 1..21: mol_type = other DNA
tcgctgctgtcctccgagtcc

SEQ ID NO: 56 - DNA, 22 bp, synthetic construct
  source 1..22: mol_type = other DNA
ggtttgcctcttctccacagac

SEQ ID NO: 57 - DNA, 22 bp, synthetic construct
  source 1..22: mol_type = other DNA
ggatctggagactgaccatcag

SEQ ID NO: 58 - DNA, 23 bp, synthetic construct
  source 1..23: mol_type = other DNA
ggtttcatagcgtgacttctccc

SEQ ID NO: 59 - DNA, 22 bp, synthetic construct
  source 1..22: mol_type = other DNA
caagaacagcctggtgaagcca

SEQ ID NO: 60 - DNA, 22 bp, synthetic construct
  source 1..22: mol_type = other DNA
acggttgctgatgaactcgcag

SEQ ID NO: 61 - DNA, 23 bp, synthetic construct
  source 1..23: mol_type = other DNA
gttcgccttcattatggactgcc

SEQ ID NO: 62 - DNA, 22 bp, synthetic construct
  source 1..22: mol_type = other DNA
aactccatccgccacaacctgt

SEQ ID NO: 63 - DNA, 21 bp, synthetic construct
  source 1..21: mol_type = other DNA
ttcttgggtcctagtgctgtt

SEQ ID NO: 64 - DNA, 23 bp, synthetic construct
  source 1..23: mol_type = other DNA
cgcttccatgtttgtccttatga

SEQ ID NO: 65 - DNA, 19 bp, synthetic construct
  source 1..19: mol_type = other DNA
ccgtggacttcttcgagcc

SEQ ID NO: 66 - DNA, 23 bp, synthetic construct
  source 1..23: mol_type = other DNA
ctgttgaatcaaactcaatgggc

SEQ ID NO: 67 - DNA, 21 bp, synthetic construct
  source 1..21: mol_type = other DNA
cttctccgtggagttttaccg

SEQ ID NO: 68 - DNA, 22 bp, synthetic construct
  source 1..22: mol_type = other DNA
gctgtcaaattgaatggtggtg

SEQ ID NO: 69 - DNA, 23 bp, synthetic construct
  source 1..23: mol_type = other DNA
cgaagccgaactctttgttatca

SEQ ID NO: 70 - DNA, 19 bp, synthetic construct
  source 1..19: mol_type = other DNA
ggcctcagtcccttgttgt

SEQ ID NO: 71 - DNA, 21 bp, synthetic construct
  source 1..21: mol_type = other DNA
atgccaaatcttgcggagaat

SEQ ID NO: 72 - DNA, 21 bp, synthetic construct
  source 1..21: mol_type = other DNA
tttgctgcgattggtgacatt

SEQ ID NO: 73 - DNA, 19 bp, synthetic construct
  source 1..19: mol_type = other DNA
ccacacgaggcgtgaactc

SEQ ID NO: 74 - DNA, 21 bp, synthetic construct
  source 1..21: mol_type = other DNA
cttcaggttacatcggggtga

SEQ ID NO: 75 - DNA, 20 bp, synthetic construct
  source 1..20: mol_type = other DNA
gaagtgccacctcgtgtgaa

SEQ ID NO: 76 - DNA, 20 bp, synthetic construct
  source 1..20: mol_type = other DNA
ggaccgtggattgccaaagt

SEQ ID NO: 77 - DNA, 22 bp, synthetic construct
  source 1..22: mol_type = other DNA
caactatcggccaactcattga

SEQ ID NO: 78 - DNA, 22 bp, synthetic construct
  source 1..22: mol_type = other DNA
gcagttcttcataagcggagat

SEQ ID NO: 79 - DNA, 23 bp, synthetic construct
  source 1..23: mol_type = other DNA
agtgaacacaatagatgtggctc

SEQ ID NO: 80 - DNA, 23 bp, synthetic construct
  source 1..23: mol_type = other DNA
ttcctgatccacctgaaacaaaa

(19) United States
(12) Patent Application Publication
(10) Pub. No.: US 2025/0332212 A1
(43) Pub. Date: Oct. 30, 2025

(54) METHOD FOR TREATING OR PREVENTING ALOPECIA

(71) Applicant: Suzhou Mingren Pharmaceutical Biotechnology Co., Ltd., Suzhou (CN)

(72) Inventors: Jianwei ZHOU, Suzhou (CN); Xuqian JIANG, Suzhou (CN); Aiping LI, Suzhou (CN)

(21) Appl. No.: 19/026,261

(22) Filed: Jan. 16, 2025

Related U.S. Application Data
(63) Continuation-in-part of application No. PCT/CN2024/099595, filed on Jun. 17, 2024.

(30) Foreign Application Priority Data
Apr. 24, 2024 (CN) 202410500681.1

(51) Int. Cl. A61K 38/08; A61P 17/14

(52) U.S. Cl. CPC A61K 38/08; A61P 17/14

(57) ABSTRACT

A method for treating or preventing alopecia including administering a patient in need thereof a pharmaceutical composition including a polypeptide. The peptide has an amino acid sequence I or II: I: FPGSDRF-Z; II: X-FPGSDRF-Z; S represents phosphorylated serine; X and Z independently represent an amino acid or an amino acid sequence; X is selected from F, (R)₉, (R)₉-F, 6-aminohexanoic acid, 6-aminohexanoic acid-F, 6-aminohexanoic acid-(R)₉, 6-aminohexanoic acid-(R)₉-F; and Z is selected from (G)ₙ-RGD or A-(G)ₙ-RGD, where n is an integer greater than or equal to 0, in the range of 0-10.

### CROSS-REFERENCE TO RELATED APPLICATIONS

This application is a continuation-in-part of International Patent Application No. PCT/CN2024/099595 with an international filing date of Jun. 17, 2024, designating the United States, now pending, further claims foreign priority benefits to Chinese Patent Application No. 202410500681.1 filed Apr. 24, 2024. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, MA 02142.

### INCORPORATION-BY-REFERENCE OF SEQUENCE LISTING

This application contains a sequence listing, which has been submitted electronically in XML file and is incorporated herein by reference in its entirety. The XML file, created on Jan. 2, 2025, is named NJYK-00401-UUS.xml, and is 71,477 bytes in size.

### BACKGROUND

The disclosure relates to the field of dermatological drugs, and more particularly to the application of JWA peptides in the preparation of anti-androgenic alopecia drugs.

Androgenetic alopecia (AGA), also known as male-pattern baldness or female-pattern alopecia, is a hair-reducing condition that occurs during and after puberty. The main pathological manifestation of AGA is the shortening of hair follicles during the anagen phase, resulting in the miniaturization of hair follicles, so that the thicker, pigmented terminal hairs are gradually replaced by fine, soft, pigmented hairs.

AGA is a complex disease, and it is widely believed that the increased incidence of AGA is related to a combination of genetic factors, androgens, and environmental factors. Currently, there are two drugs approved by the US Food and Drug Administration (FDA) for the treatment of AGA: topical minoxidil and oral finasteride.

Minoxidil has potent vasodilatory properties and by increasing blood circulation, inducing vasodilation as well as overexpression of vascular endothelial growth factor (VEGF), minoxidil promotes faster and thicker hair growth, prolongs the anagen phase, and increases mitosis in keratinized cells of the hair matrix. However, minoxidil can cause adverse reactions such as itchiness of the scalp, dermatitis, and localized hirsutism. Finasteride is a type II 5α-reductase inhibitor, can irreversibly bind to the enzyme, reducing double hydrogen testosterone (DHT) by more than 60% in serum and scalp. In addition, finasteride induces the conversion of resting phases into growing phases in the hair cycle, but carries the risk of sexual dysfunction and depression. Both drugs are expensive, dependent after use, and once discontinued they re-enter the alopecia areata phase. In summary, the currently available treatments for AGA are very limited and all have varying degrees of side effects that may cause secondary damage to the patient's body and mind.

JWA gene, also known as ARL6IP5 (GenBank: AF070523, 1998), is a typical and broad-spectrum environmental response gene, and is also an aging-related gene. The gene is widely involved in cellular responses to a variety of environmental physical and chemical stimuli, such as oxidative stress, heat stress, etc. JWA knockout mice showed aging phenotypes, such as weight loss, shortened lifespan, skin atrophy, and hair loss, and JWA-deficient cells also showed early aging phenotypes. The previous study also found that JWA is an important molecule for renewal and post-damage repair of intestinal epithelium, and its mechanism is not only related to anti-oxidative stress and repair of DNA damage, but also to activation of small intestinal addictive fossa stem cell proliferation and differentiation; conditional knockout of the JWA gene in small intestinal epithelium resulted in premature senescence phenotypes similar to those seen in the systemic knockout mice of the gene. JP1 (sequence: FPGSDRFGGGG-RGD (SEQ ID NO: 6), S-site phosphorylation) is a small molecule peptide designed and synthesized from the coding sequence of JWA protein based on the results of previous basic research.

Previous studies have demonstrated that JP1 can perform biological functions similar to those of the JWA protein and its mechanism of action is related to the activation of the MAPK signaling pathway, which suggests that the JWA peptide can achieve some of the biological functions of the JWA protein through a similar mechanism.

### SUMMARY

To solve the aforesaid problems, the first objective of the disclosure is to provide an application of a JWA peptide in the preparation of an anti-androgenic alopecia drug, which is administered systemically by injection or used topically on the skin, plays the role of promoting proliferation and activation and other effects on the skin's hair follicle stem cells, promote the growth of hair, and effectively reverse the inhibitory effect of androgens on the hair follicle cells, so as to provide a new possibility of clinical use of drugs for the treatment of androgenic alopecia.

Specifically, the disclosure provides a method for treating or preventing alopecia comprising administering a patient in need thereof a pharmaceutical composition comprising a polypeptide, the peptide having an amino acid sequence I or II: I: FPGSDRF (SEQ ID NO: 1)-Z; II: X-FPGSDRF (SEQ ID NO: 1)-Z;
S represents phosphorylated serine;
X and Z independently represent an amino acid or an amino acid sequence;
X is selected from F, (R)₉ (SEQ ID NO: 2), (R)₉-F (SEQ ID NO: 3), 6-aminohexanoic acid, 6-aminohexanoic acid-F, 6-aminohexanoic acid-(R)₉ (SEQ ID NO: 2), 6-aminohexanoic acid-(R)₉-F (SEQ ID NO: 3); and
Z is selected from (G)ₙ-RGD or A-(G)ₙ-RGD (SEQ ID NO: 4), where n is an integer greater than or equal to 0, in the range of 0-10.

In a class of this embodiment, the alopecia comprises androgenetic alopecia.

In a class of this embodiment, the pharmaceutical composition is configured to target hair follicle tissues, promote a growth of hair follicles and hair rods, shorten a resting period of hair follicles, prolong a growth period of hair follicles, and significantly reverse an inhibition of hair growth by androgens.

In a class of this embodiment, the pharmaceutical composition is configured to target integrin molecules into hair follicle cells and upregulate an expression of integrin molecules in a hair follicle cell membrane; and the integrin molecules comprise αvβ1.

In a class of this embodiment, the pharmaceutical composition is configured to promote an expression of a nuclear transcription factor SP1 by activating a MEK/ERK/E2F1 signaling axis, which in turn activates a Wnt/β-catenin signaling pathway in hair follicle stem cells.

In a class of this embodiment, the polypeptide comprises an acetylated N-terminal and an amidated C-terminal.

In a class of this embodiment, an amino acid sequence of the polypeptide is one of SEQ ID NO: 5 to SEQ ID NO: 27.

In a class of this embodiment, each amino acid in the FPGSDRF (SEQ ID NO: 1) sequence is either L-type or D-type.

In a class of this embodiment, the pharmaceutical composition further comprises a pharmaceutically acceptable excipient.

In a class of this embodiment, a dosage form of the pharmaceutical composition is injectable or for external use.

The polypeptides have therapeutic effects on androgenetic alopecia, which is achieved by targeting integrin molecules directly to the hair follicle cells and entering the cells to regulate the proliferation of hair follicles and promote the growth of hair, etc., thus shortening the resting phase and prolonging the anagen phase of hair follicle, increasing the volume of hair rods and hair follicles, increasing the expression level of integrin molecules in the target cells αvβ1, and precisely activating the signaling pathway MEK/ERK/E2F1/SP1/Wnt10a/10b/β-catenin of the hair follicle stem cell. Therefore, the polypeptides have a good potential for use in the preparation of drugs targeting androgenetic alopecia.

### BRIEF DESCRIPTION OF THE DRAWINGS

FIGS. 1A-1C show the design and initial screening results of the JP1-promoted AGA mouse hair growth model in Example 1 of the disclosure. FIG. 1A is a schematic diagram of the animal model. FIG. 1B is photographs of skin appearance on the back of mice. FIG. 1C is a trend graph of body weight of mice.

FIGS. 2A-2C show experimental results of JP1 promoting skin color transformation in AGA model mice in Example 2 of the disclosure. FIG. 2A is a schematic diagram of skin color score. FIG. 2B is a statistical graph of skin color scores of mice. FIG. 2C is a summary table of P-values between groups at each time period.

FIGS. 3A-3D show graphs of the experimental results of JP1 promoting the advancement of hair follicles into the anagen phase in the skin of AGA model mice in Example 3 of the disclosure. FIG. 3A shows a longitudinal section diagram of H&E staining of hair follicles. FIG. 3B shows a cross-section diagram of H&E staining of hair follicles. FIG. 3C shows a statistical graph of dermal layer thickness in mice. FIG. 3D shows a statistical graph of total number of hair follicles in mice.

FIG. 4 shows the experimental results of the stability assessment of JP1 in skin-coating solvents in Example 4 of the disclosure, in which a summary of the main peak area and the percentage of the peak area of each JP1 group is shown.

FIG. 5 shows experimental results of JP1 targeting into the hair follicle site and its half-life assessment in skin tissues in Example 5 of the disclosure, specifically, immunofluorescence staining of FITC-JP1 into the hair follicle pathway and H&E staining of the corresponding region.

FIGS. 6A-6E show the experimental results of JP1 promoting hair growth in a dose-dependent manner in Example 6 of the disclosure. FIG. 6A shows a diagram of animal model design. FIG. 6B shows appearance diagrams of dorsal skin color changes at different time periods during the model in each group of mice. FIG. 6C shows a graph of body weight of model mice. FIGS. 6D-6E show back skin color score of mice and summary of P-value.

FIGS. 7A-7E show the experimental results of JP1 in Example 7 of the disclosure for extending the time when the hair follicle is in anagen phase. FIG. 7A shows a longitudinal section of H&E staining of hair follicles. FIG. 7B shows a statistical graph of dermal layer thickness in mice. FIG. 7C shows cross-section of H&E staining of hair follicles on day 11. FIG. 7D shows a statistical graph of total number of hair follicles in mice on day 11. FIG. 7E shows a statistical graph of hair follicle bulb diameter on day 11.

FIGS. 8A-8C show experimental results of JP1 exerting a pro-hair follicle growth effect by activating the Wnt signaling pathway in Example 8 of the disclosure. FIG. 8A shows AR and SRD5A2 mRNA expression levels in the skin of mice in the control, model, JP1 treatment and minoxidil groups. FIG. 8B shows a KEGG enrichment map of up-regulated genes in 1% JP1 vs model group. FIG. 8C shows gene enrichment by GSEA analysis.

FIGS. 9A-9E show experimental results of JP1 activating β-catenin expression in skin tissue in Example 9 of the disclosure. FIG. 9A shows β-catenin, LEF1 mRNA expression level. FIG. 9B shows β-catenin protein expression level detected by protein immunoblotting assay. FIG. 9C shows quantitative analysis of β-catenin protein expression. FIG. 9D shows representative pictures of each group of β-catenin detected by immunofluorescence staining. FIG. 9E shows a statistical graph of fluorescence intensity.

FIGS. 10A-10C show the experimental results of JP1 in Example 10 of the disclosure for increasing the expression level of β-catenin via Wnt10a/Wnt10b. FIG. 10A shows screening of Wnt ligands regulating β-catenin and detection of mRNA expression level. FIG. 10B shows correlation between JWA mRNA and Wnt10a mRNA. FIG. 10C shows correlation between JWA mRNA and Wnt10b mRNA.

FIGS. 11A-11D show the experimental results of the up-regulation of Wnt10a/Wnt10b expression by JP1 possibly through SP1/MYC in Example 11 of the disclosure. FIG. 11A shows immunoblotting to detect Wnt10a/Wnt10b protein expression levels in each group. FIG. 11B shows relative quantitative analysis of Wnt10a protein expression in each group. FIG. 11C shows relative quantitative analysis of Wnt10b protein expression in each group. FIG. 11D shows online tool to predict transcription factors for JP1 activation of Wnt10a/Wnt10b.

FIGS. 12A-12E show experimental results of JP1 promoting increased SP1 expression in Example 12 of the disclosure. FIG. 12A shows SP1 and MYC mRNA expression levels. FIG. 12B shows immunoblotting to detect SP1 protein expression level. FIG. 12C shows relative quantitative analysis of SP1 protein expression. FIG. 12D shows representative pictures of SP1 in each group detected by immunohistofluorescence staining. FIG. 12E shows a statistical graph of fluorescence intensity.

FIGS. 13A-13E show the experimental results of JP1 activating the Wnt pathway through the E2F1-SP1 axis in Example 13 of the disclosure. FIG. 13A shows prediction of SP1 upstream transcription factors using 3 online tools and intersections of transcription factors were taken. FIG. 13B shows qPCR detection of FOXI1 mRNA expression level. FIG. 13C shows qPCR detection of FOXD3 mRNA expression level. FIG. 13D shows qPCR detection of E2F1 mRNA expression level. FIG. 13E shows correlation between SP1 mRNA and E2F1 mRNA, data from GSE212301.

FIGS. 14A-14B show experimental results of JP1 acting through the MEK/ERK/E2F1 axis in Example 14 of the disclosure. FIG. 14A shows protein immunoblotting to detect changes in protein levels of MAPK signaling pathway related molecules p-c-Raf, p-MEK, p-ERK and E2F1. FIG. 14B shows Image J for protein gray value analysis.

FIGS. 15A-15D show the experimental results that inhibition of SP1 by MTA in Example 15 of the disclosure can block the hair follicle growth-promoting effect of JP1 on AGA model mice. FIG. 15A shows the model design scheme. FIG. 15B shows a graph of body weight change of mice during the experiment. FIG. 15C shows the appearance of hair loss areas in the skin of mice during the experiment. FIG. 15D shows a graph of skin color scores of mice in each group.

FIGS. 16A-16E show the experimental results of histomorphometric changes in hair follicle growth promotion in AGA model mice by MTA inhibition of SP1 blocking JP1 in Example 16 of the disclosure. FIG. 16A shows longitudinal section of hair follicle H&E staining. FIG. 16B shows relative ratio of dermal layer thickness of mouse skin in each group on day 12. FIG. 16C shows statistics of the diameter of the hair bulb part of the hair follicle in mice on day 12. FIG. 16D shows H&E-stained transverse section of hair follicle. FIG. 16E shows a statistical graph of the total number of hair follicles in mouse skin on day 12.

FIGS. 17A-17C show experimental results of the effect of MTA blocking JP1 on the expression of SP1 downstream molecules in Example 17 of the disclosure. FIG. 17A shows immunoblotting to detect the expression changes of key molecules in the JP1 mechanistic pathway at the end of the model (day 12). FIG. 17B shows changes in protein levels of SP1, Wnt10a, Wnt10b and β-catenin in mouse skin on day 4. FIG. 17C shows changes in protein levels of SP1, Wnt10a, Wnt10b and β-catenin in mouse skin on day 8.

FIGS. 18A-18D show experimental results of JP1 targeting integrin αvβ1 into hair follicle tissue in Example 18 of the disclosure. FIG. 18A shows mRNA expression levels of integrin α and integrin β subunits that can bind to RGD in skin tissues of control and AGA mice. FIG. 18B shows effect of JP1 intervention on the mRNA expression levels of individual integrin α & β subunits. FIG. 18C shows immunoblotting for protein expression levels of integrin αv and β1. FIG. 18D shows integrin αv and β1 gray values through Image J statistics.

FIGS. 19A-19D show a representative graph of the immunofluorescence detection results of integrin αv and β1 in Example 19 of the disclosure. FIG. 19A shows representative maps of immunofluorescence staining tissue distribution and expression level of integrin αv in each group. FIG. 19B shows Image J statistics of fluorescence intensity of integrin αv in each group and comparison of their differences. FIG. 19C shows a representative graph of immunofluorescence staining tissue distribution and expression level of integrin β1. FIG. 19D shows Image J statistics of fluorescence intensity of integrin β1 in each group and comparison of their differences.

FIG. 20 shows the molecular mechanism of JP1 of the disclosure for promoting hair growth in the TP-induced AGA model.

### DETAILED DESCRIPTION

To further illustrate the disclosure, embodiments detailing a method for treating or preventing alopecia are described below. It should be noted that the following embodiments are intended to describe and not to limit the disclosure. The materials, methods, experimental model conditions, etc. used in each example are attached to each embodiment, and without special instructions, the materials (e.g., commercially available products) and the experimental methods used are all conventional materials and methods.

### EXAMPLE 1

The sequence of JP1 employed in this example is FPGSDRF-RGD (SEQ ID NO: 28), in which the amino acid S is phosphorylated; the conformation of each amino acid of the sequence FPGSDRF (SEQ ID NO: 1) is either L-type or D-type. The following examples employ the same JP1 as this example and will not be repeated below.

The example studies the effect of JP1 on hair growth.

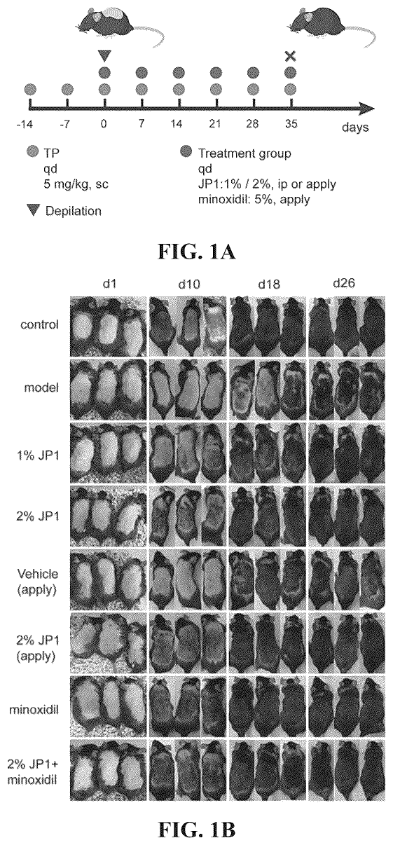

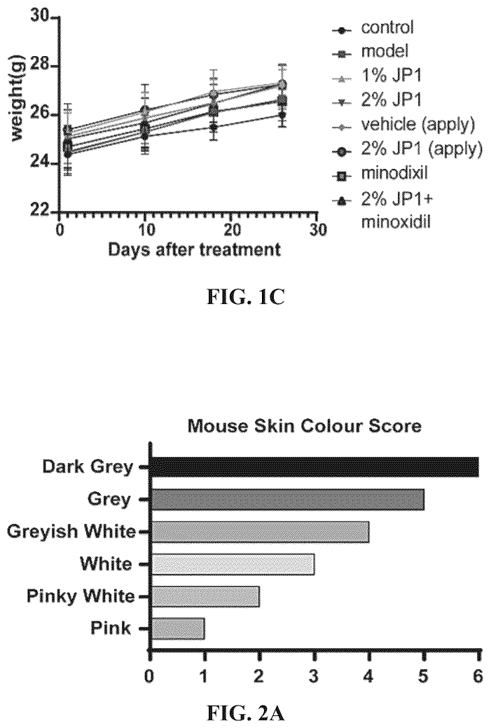

C57BL/6 mice were selected for modeling in this example according to the first design scheme (FIG. 1A). For more details, see “Animal model I” of “1. TP-induced AGA model” in “Experimental Methods” below.

### Experimental Results:

(1) The skin on the back of control mice turned black on the 10th day, while no change occurred in the model group, suggesting successful modeling.
(2) JP1, whether injected intraperitoneally or applied to the skin, promoted hair growth.
(3) Combined treatment of 2% JP1 and minoxidil had a synergistic effect, which was stronger than that of JP1 or minoxidil alone in the treatment group (FIG. 1B)
(4) The difference in body weight gain during the mouse modeling between groups was not statistically significant (FIG. 1C, P>0.05).

The results of the example show that JP1 promoted hair growth in AGA mice.

### EXAMPLE 2

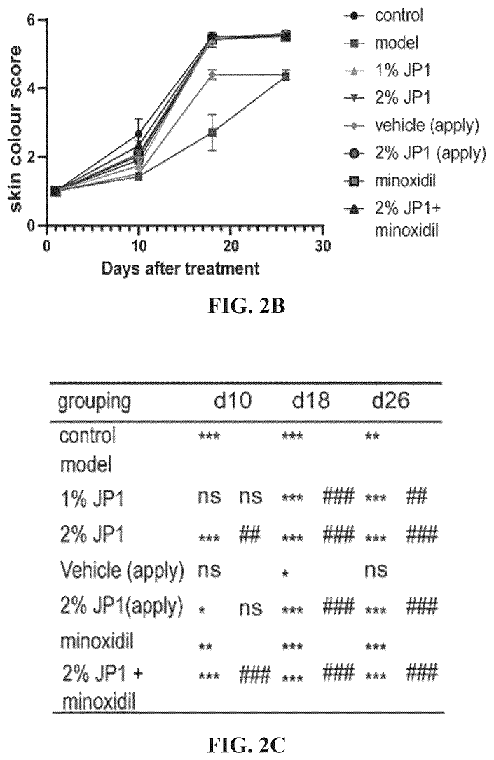

The example scored the skin color change in the AGA experimental area of model mice of Example 1, and the results are shown in FIGS. 2A-2C.

FIG. 2A shows the reference standard for scoring the color of the dorsal depilated area of control mice.

### Experimental Results:

(1) The hair growth index of the 2% JP1 group was significantly higher than that of the model group (P<0.001), and the hair growth index of the 2% JP1 (apply) group was also higher than that of the model group (P<0.05), but the skin color of the mice in the 1% JP1 group had just begun to change, which was statistically not significant when compared with the model group (P>0.05). Note: The 1% JP1 and 2% JP1 groups were administered by intraperitoneal injection, and the 2% JP1 (apply) group was administered by topical skin coating, the same as below.
(2) The hair growth index of the 2% JP1 (apply) group was also higher than that of the solvent group (vehicle (apply) group), indicating that the solvent alone did not have a promotional effect on hair growth (FIGS. 2B-2C).

The results of this example show that JP1 significantly accelerated skin color change on the back of AGA mice.

### EXAMPLE 3

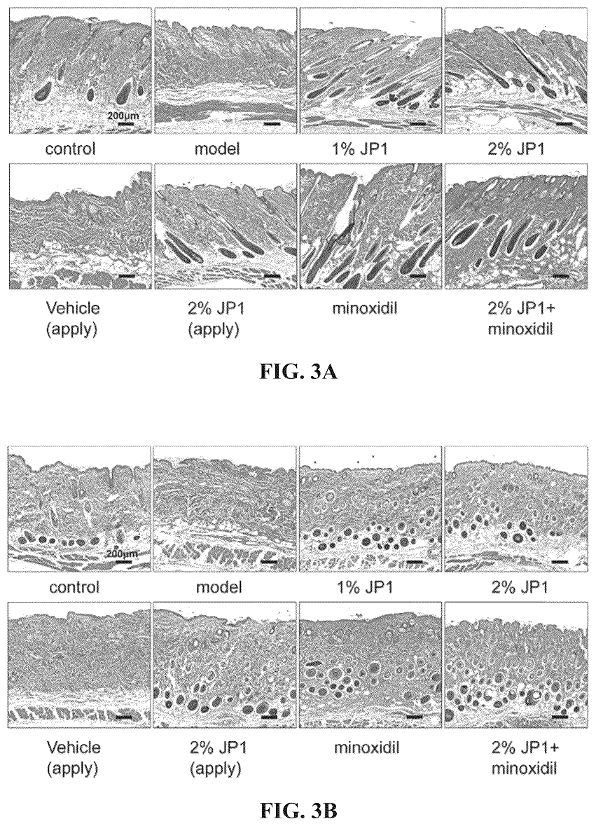

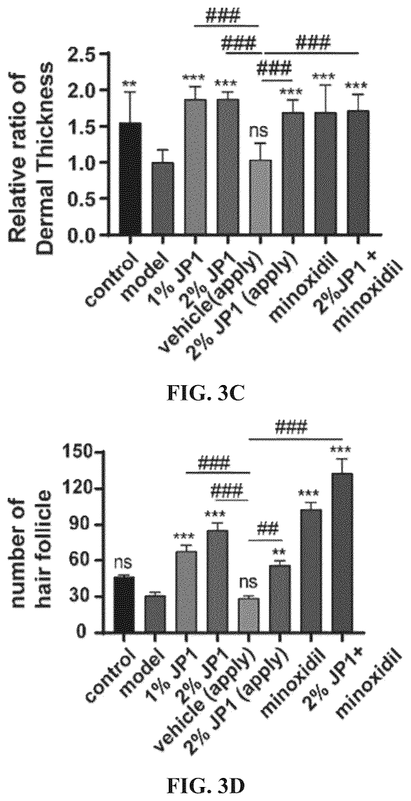

In this Example, the dermal layer thickness, number and size of hair follicles were observed after H&E staining of transverse and longitudinal sections of the skin tissues in the experimental area of the AGA model mice of Example 1, and the results are shown in FIGS. 3A-3D.

### Experimental Results:

(1) The longitudinal section results showed increased dermal layer thickness and longer hair follicle length in each JP1 treatment group compared to the model and solvent groups (FIG. 3A). The thickness of the dermal layer was correlated with the proliferative cycle of the hair follicle, which was thicker when the hair follicle was in the anagen phase and thinner as the hair follicle gradually entered the regressive phase; the model group and the solvent control group were in the resting phase, whereas the other treatment groups had already entered the anagen phase (FIG. 3C).
(2) Cross-sectional results showed that the total number of hair follicles was greater in each treatment group than in the model and solvent control groups (P<0.001, FIG. 3B and FIG. 3D). The total number of hair follicles was greater in the combination of 2% JP1 and minoxidil than in their respective effects alone (FIG. 3B and FIG. 3D).

The results of this example show that JP1 was able to shorten the resting phase of the hair follicle, allowing the hair follicle to enter the anagen phase quickly, and was comparable to the effect of minoxidil.

### EXAMPLE 4

The results of Example 1 demonstrated that the JP1 skin-coating group promoted hair growth in AGA mice and had a similar effect to the intraperitoneal injection group.

This example further explored the stability of JP1 in the skin-coating solvent. In this example, the solvent group and JP1 group were designed, and the content and molecular weight of JP1 in the skin-coating solvent were detected using high performance liquid chromatography on days 1, 3, 7, and 14, respectively, after the solution was formulated for evaluation. The prepared solutions were placed in a refrigerator at 4° C. The details of the experiments are shown in “II. JP1 Stability Experiments” of “Experimental Methods” below.

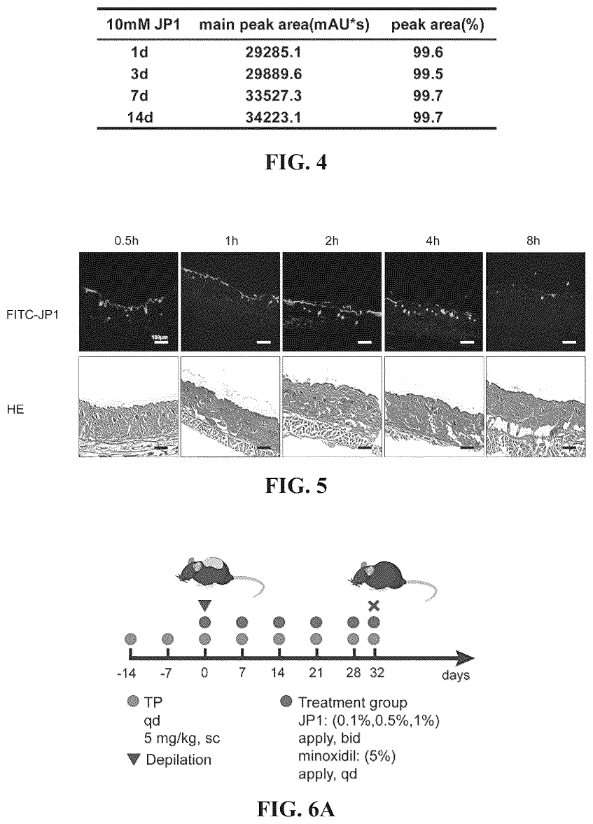

The results are shown in FIG. 4: no JP1 degradation was detected in the skin-coated solvent for 14 days, and the percentage of the main peak area was nearly 100%, indicating that except for the impurities in the solvent, the peak area of the substance produced by the degradation of the drug to be tested was less than 0.5 mAU, and no JP1 degradation was detected.

The results of this example show that JP1 has good stability.

### EXAMPLE 5

In this Example, to elucidate the mechanism of JP1 penetrating the skin and acting on the hair follicle, a 14-day AGA modeling was performed according to the model design strategy of Example 1 above. Thereafter, a 1% FITC-JP1 solution was applied to the dorsal depilated area of the mice in a light-avoidance environment. Five time points were set at 0.5, 1, 2, 4 and 8 h for observation. The fresh skin tissue was made into frozen sections, and the penetration pathway of JP1 in the skin tissue and the amount of aggregation in the hair follicles were observed under an inverted fluorescence microscope. The details of the experiments are shown in “III. JP1 Permeability Experiments” of “Experimental Methods” below.

The results are shown in FIG. 5: compared with the skin structure shown by H&E staining, the drug basically covered the surface of the skin at 0.5 h, and part of the drug had already entered into the deep layer of the skin, and then gradually entered into the hair follicle downward, reaching the peak at 4 h, and the fluorescence brightness was obviously weakened at 8 h, which indicated that JP1 was able to penetrate into the skin and enter into the follicle tissue in a targeted manner.

The results of this example show that JP1 can penetrate the skin and target the hair follicle tissue, and has good transdermal properties.

### EXAMPLE 6

The results of the animal model in Example 1 have confirmed that JP1 either intraperitoneally injected or topically applied to the skin can promote hair growth. To investigate whether there is a dose-effect relationship between JP1 topical application of skin on AGA mice, this Example constructed an AGA mouse model and then intervened with different doses of JP1 topical application of skin.

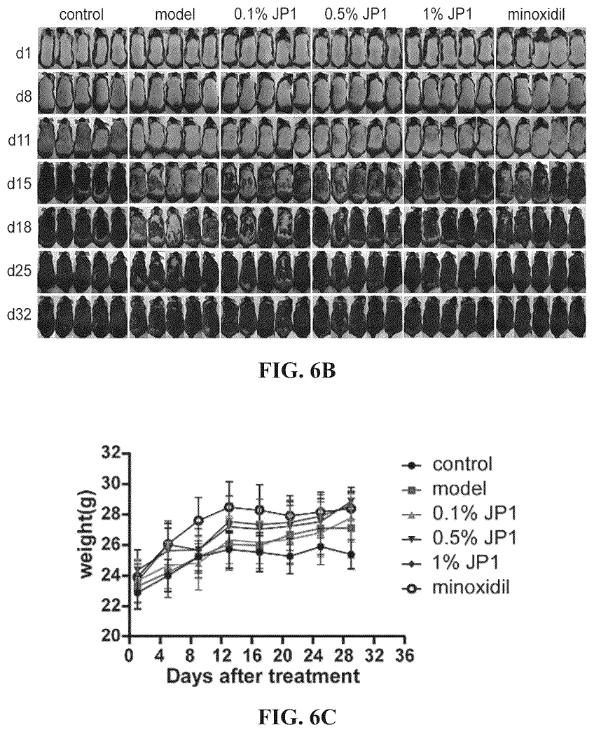

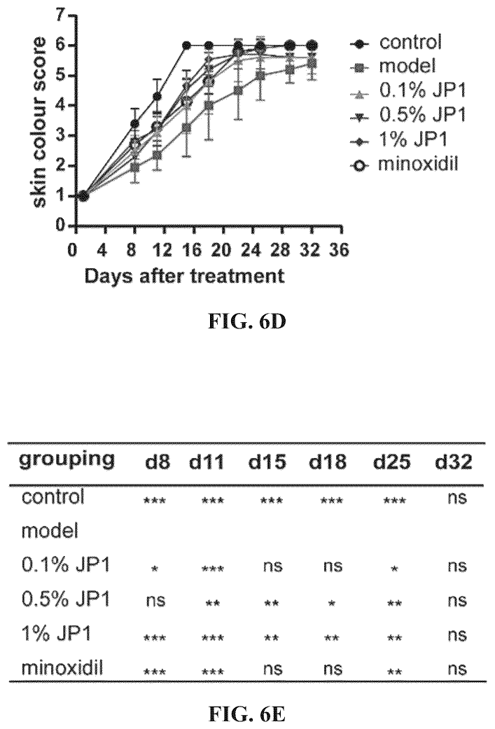

In the experiment exploring the transdermal properties of JP1 (Example 5), it has been found that JP1 had the highest amount of fluorescence aggregation of FITC connected to JP1 at the hair follicle site at 4 h after administration, while JP1 significantly decreased at 8 h. To prolong the action time of JP1, the design scheme of the animal model in this example adjusted the once-a-day of the first model to twice-a-day administration with an interval of 6 h (FIG. 6A). For details of the experiments, please refer to “Animal model II” of “1. TP-induced AGA model” in “Experimental Methods” below.

The results showed that on the 11th day after continuous intervention, the skin color of mice in the model group appeared orange, while the JP1 and minoxidil groups had changed to gray; the difference in the skin color of the mice in each group became more obvious on the 15th day; on the 18th day, the 1% JP1 group had the highest hair coverage, followed by 0.5% JP1, and the lowest was 0.1% JP1 (FIG. 6B). Skin color scores also exhibited color differences (FIG. 6D and FIG. 6E). Meanwhile, there was no statistically significant difference between the body weights of the mice in each group during the experiment (FIG. 6C). These results suggest that JP1 skin-coating administration promotes the regeneration of AGA hair follicle induced by TP with a significant dose-effect relationship.

The results of this exemplary suggest that JP1 dose-dependently promotes hair growth.

### EXAMPLE 7

In this Example, longitudinal H&E staining of hair follicles was performed on skin tissue samples from the model mice in Example 6.

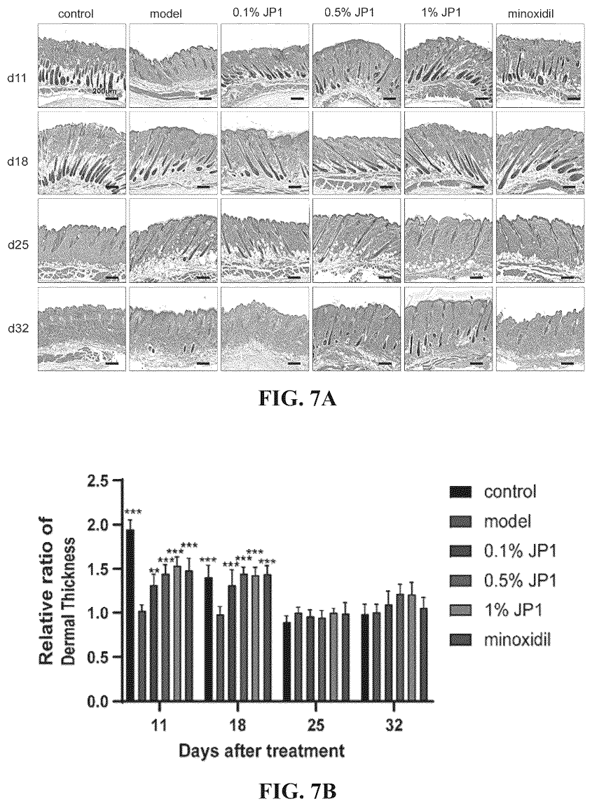

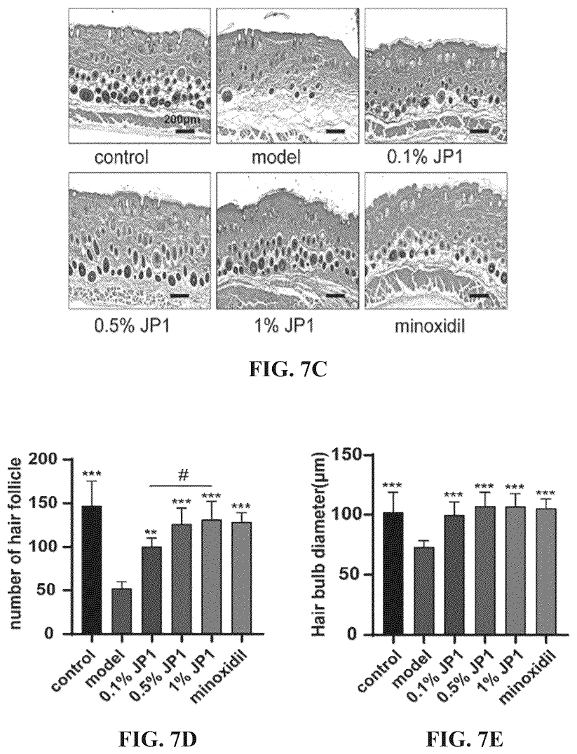

The results are showed in FIGS. 7A-7E: on day 11 the model group just entered the anagen phase, while the JP1 intervention group was in the mid-growth phase, the hair follicles were longer and the bulb of the hair follicles was larger, and the hair bulb was the largest in the 1% JP1 group; on day 18 the groups were in the anagen phase; on day 25 the groups were gradually shifted to the regression phase; on day 32 the model group entered the resting phase, and the hair follicles of the medium and high dose JP1 group entered the anagen phase again. The changes in dermal layer thickness of the skin were consistent with the results of longitudinal sectioning. The results of hair follicle transverse sections showed that the total number of hair follicles in the JP1 intervention group increased significantly and dose-dependently compared with the model group; the diameter of the hair bulb was also significantly larger than that of the model group.

The results of this example showed that the difference in the growth cycle of hair follicles in the groups after JP1 or minoxidil intervention was obvious; both drugs could accelerate the entry of hair follicles into the anagen phase and shorten the resting phase, and were able to prolong the time that the hair follicles were in the anagen phase. This is also pathological evidence for the dose-dependent hair growth promotion of JP1.

### EXAMPLE 8

In this Example, model mouse skin tissues in Example 6 were used.

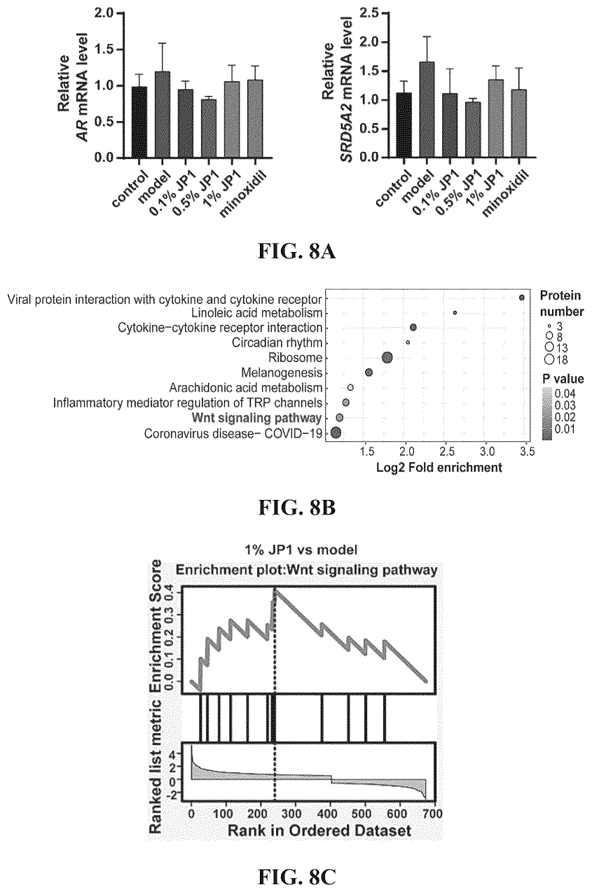

To elucidate the molecular mechanism of JP1 promoting hair follicle growth, in this Example, the mRNA levels of AR and SRD5A2 of the skin tissues of each model mouse were examined, and the results showed that there was no statistically significant difference in the mRNA levels between the groups (P>0.05 in FIG. 8A).

To explore the mechanism of the hair growth-promoting effect of JP1 at the protein level, the skin tissues of mice in the model group and 1% JP1 group were subjected to high-throughput sequencing of proteomics and protein phosphorylation modification in this example; the results of KEGG signaling pathway analysis of the sequencing data showed that the differences between the two groups were mainly enriched in the Wnt signaling pathway (FIG. 8B); the results of the GSEA analysis also showed that the Wnt/β-catenin signaling pathway was enriched in the skin tissues of JP1-treated mice (FIG. 8C).

The results of this example show that JP1 exerts a pro-follicular growth effect by activating the Wnt signaling pathway.

### EXAMPLE 9

This example validates the regulatory effect of JP1 on β-catenin.

β-catenin is a critical indicator of the Wnt signaling pathway and a key point in this signaling pathway that controls hair follicle formation.

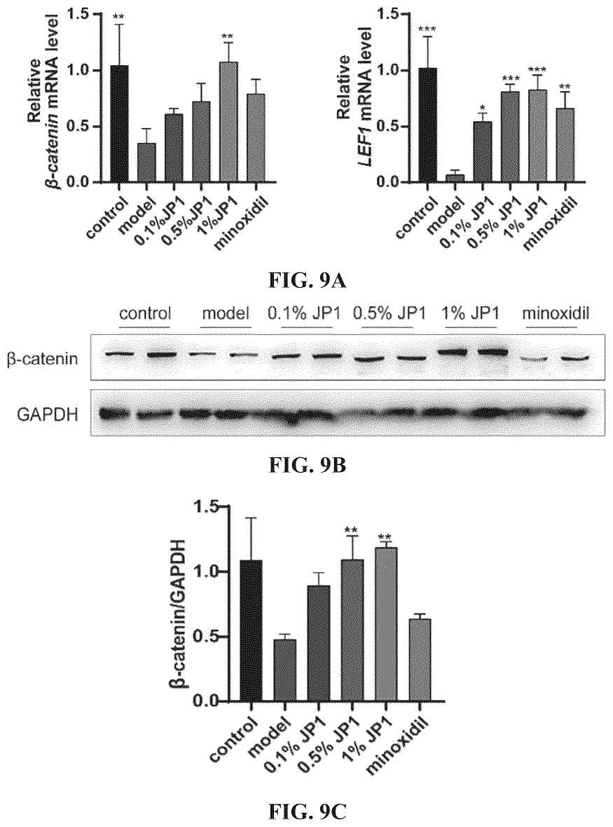

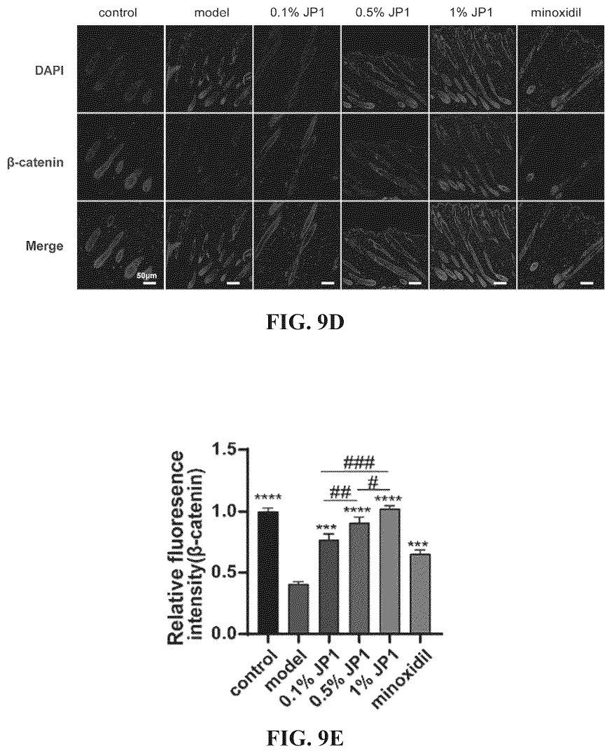

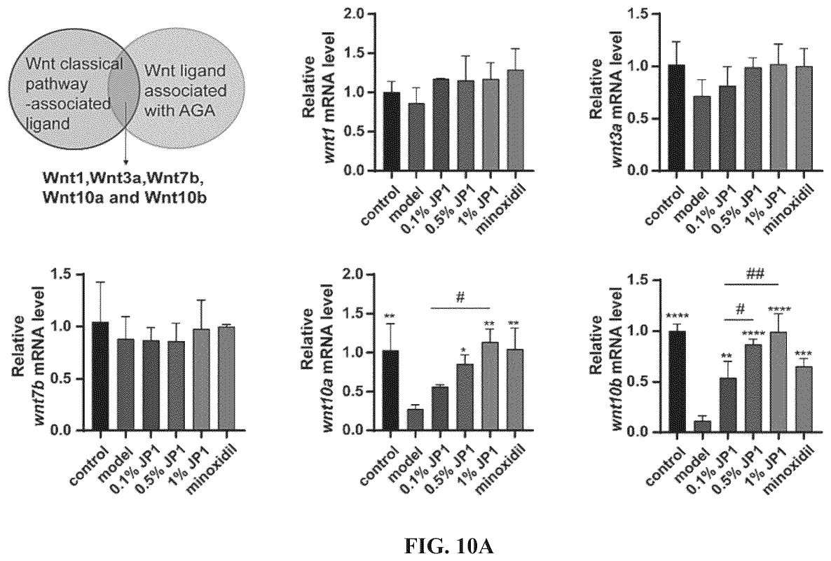

The results showed (FIGS. 9A-9E): as shown by the detection results of mRNA level of β-catenin and its nuclear transcription factor Lef1 in skin tissues, compared with the model group, the expression of β-catenin and Lef1 increased in the JP1 group (FIG. 9A). As shown by the results of protein immunoblotting experiments, β-catenin expression was increased after JP1 intervention, and there was a dose-effect relationship; minoxidil could also act through this pathway, but the effect was slightly weaker than that of the JP1 group (FIGS. 9B-9C). The results of immunohistofluorescence staining showed a trend consistent with the results of protein immunoblotting experiments (FIGS. 9D-9E).

### EXAMPLE 10

This example verified that JP1 regulated the upstream Wnt ligand of β-catenin.

The Wnt family can be divided into 2 types, one is involved in the β-catenin-mediated classical signaling pathway, including Wnt1, Wnt2, Wnt2b, Wnt3, Wnt3a, Wnt7a, Wnt7b, Wnt8, Wnt8b, Wnt10A, and Wnt10b; and the other is a non-classical signaling pathway that is not dependent on β-catenin, including Wnt4, Wnt5a, Wnt6, and Wnt11. The main Wnt ligands that have been involved in androgenetic alopecia include Wnt1, Wnt3a, Wnt4, Wnt5a, Wnt7b, Wnt10a, and Wnt10b.

To illustrate the regulation of JP1 on the upstream Wnt ligand of β-catenin, this example intersected classical signaling pathway-related Wnt ligands and AGA-related Wnt ligands, and screened out the related Wnt ligands, including Wnt1, Wnt3a, Wnt7b, Wnt10a, and Wnt10b. In this example, qPCR was used to detect the mRNA expression of these Wnt molecules in the skin tissue of the mouse model in Example 6.

### The Results Showed:

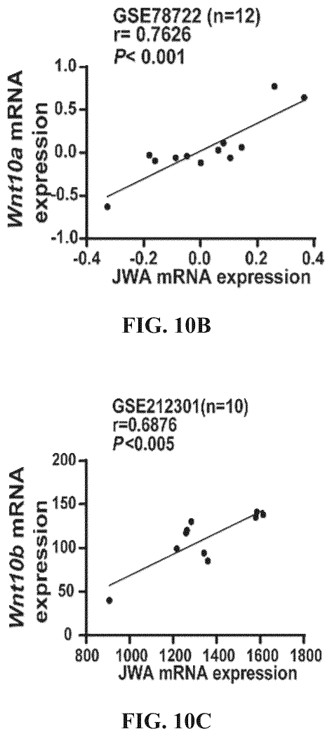

(1) in the JP1 group, the expression of both Wnt10a and Wnt10b was elevated with a dose-effect relationship (P<0.05); while the mRNA levels of the other Wnt molecules were not statistically significant among the groups (P>0.05, FIG. 10A).
(2) Further analysis of the correlation between Wnt10a/Wnt10b and JWA in the tissues of alopecia areata of AGA patients in public databases revealed that JWA mRNA showed a significant positive correlation with the Wnt10a mRNA expression level (P<0.001, r=0.7626, FIG. 10B), and JWA mRNA and Wnt10b mRNA expression levels also showed a significant positive correlation (P<0.005, r=0.6876, FIG. 10C).

The results of this example show that JP1 increases β-catenin expression level through Wnt10a/Wnt10b.

### EXAMPLE 11

This example predicts that JP1 regulates upstream transcription factors of Wnt/β-catenin.

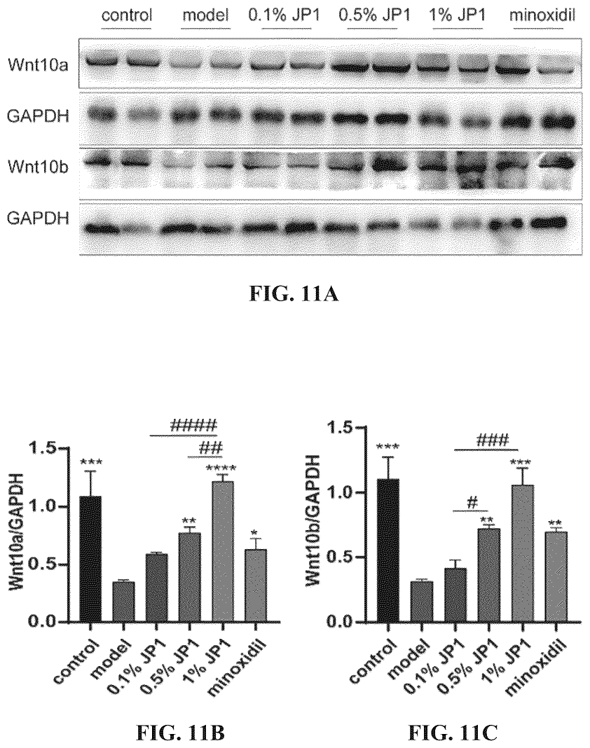

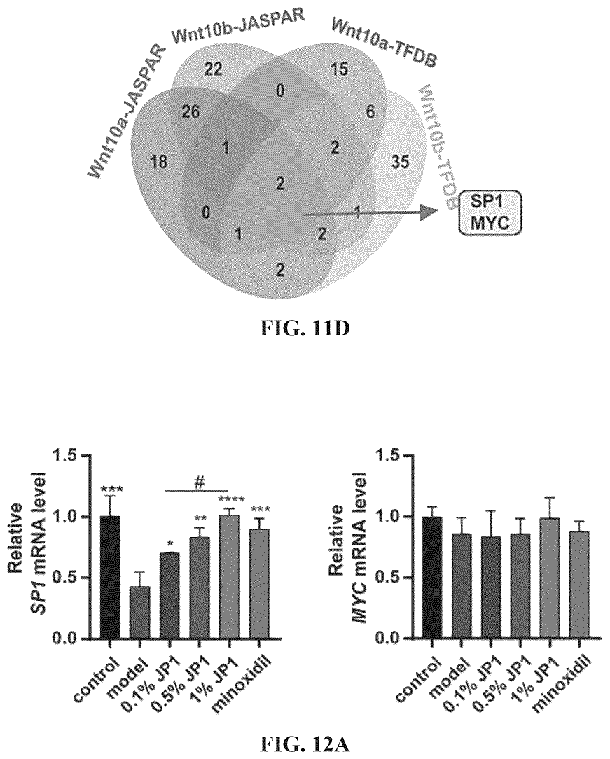

Western blotting experiment on the skin tissues of the model mice in Example 6 was conducted, and the results confirmed that JP1 significantly up-regulated the protein expression of Wnt10a and Wnt10b in a dose-dependent manner (FIGS. 11A-11C).

To demonstrate the mechanism by which JP1 up-regulates Wnt10a/Wnt10b expression, the example used two online tools JASPAR (https://jaspar.elixir.no/) and Animal TFDB (https://guolab.wchscu.cn/AnimalTFDB4//#/) to predict the Wnt10a/Wnt10b transcription factors and took the intersection thereof to find two candidate transcription factors, SP1 and MYC (FIG. 11D). The results suggested that JP1 might upregulate Wnt10a/Wnt10b expression through SP1/MYC.

### EXAMPLE 12

This example verifies that SP1 is a JP1 transcription factor that regulates Wnt/β-catenin.

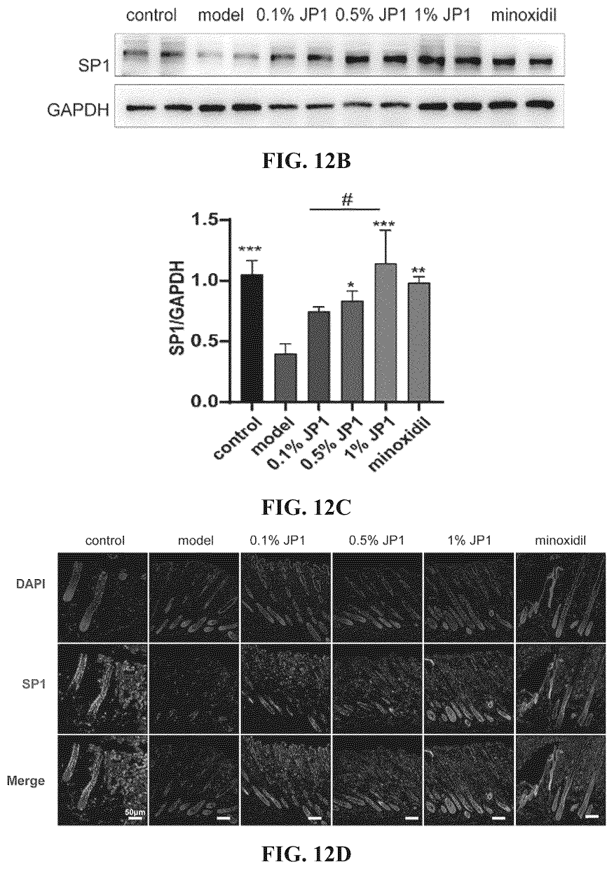

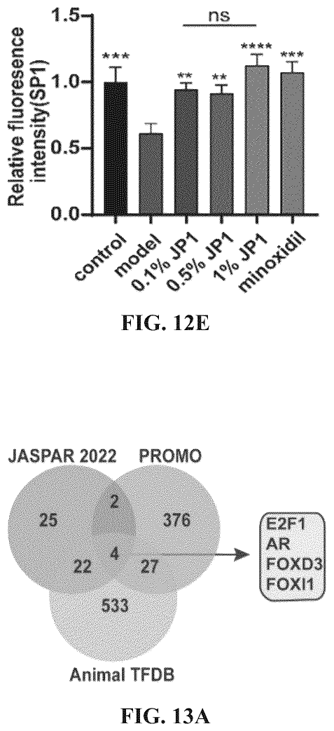

Based on the prediction results of the online tool of Example 11, qPCR was used to detect the mRNA levels of SP1 and MYC in the skin tissue of the mouse model in Example 6, and the results showed that:
(1) SP1 mRNA showed high expression in the JP1 group, and the expression level in the 1% JP1 group was significantly higher than that in the 0.1% JP1 group (P<0.05), whereas the difference in the MYC mRNA level between groups was not statistically significant (P>0.05), suggesting that the nuclear transcription factor for activation of Wnt10a and Wnt10b by JP1 may be SP1 (FIG. 12A).
(2) The results of SP1 protein level detection by immunoblotting showed that the expression level of SP1 showed an elevated trend in the JP1 group (FIG. 12B and FIG. 12C).
(3) Similarly, the immunofluorescence results were consistent with the protein immunoblotting results (FIG. 12D and FIG. 12E).

The results of this example show that JP1 promotes increased SP1 expression.

### EXAMPLE 13

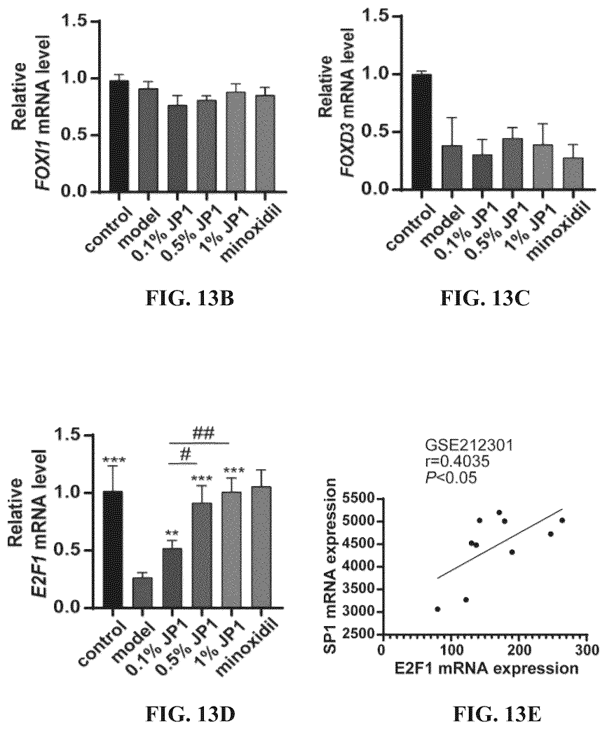

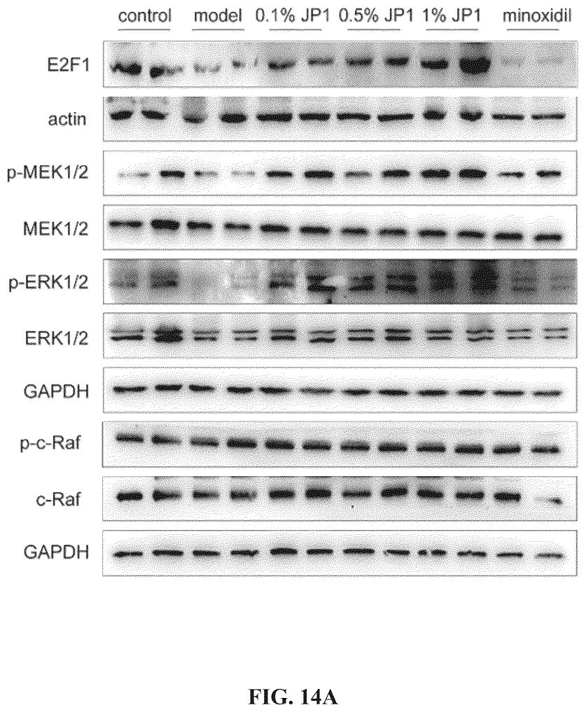

To further demonstrate that JP1 activates the upstream regulatory mechanism of SP1 expression, this example used three online tools for transcription factor prediction: JASPAR (https://jaspar.elixir.no/), PROMO (https://alggen.lsi.upc.es/cgibin/promo_v3/promo/promoinit.cgi?dirDB=TF_8.3), Animal TFDB (https://guolab.wchscu.cn/AnimalTFDB4//#/) (FIG. 13A). Four transcription factors that can bind to the SP1 promoter region were obtained by taking the intersection of the predicted data: E2F1, AR, FOXD3, and FOXI1 (FIG. 13A).

In this example, the mRNA levels of the four transcription factors mentioned above in the skin tissue of the mouse model in Example 6 were further examined, and the results showed that:
(1) The mRNA level expression of E2F1 was elevated after JP1 intervention (P<0.01), and the expression of other molecules did not change significantly (P>0.05), suggesting that E2F1 regulates SP1 and thus induces changes in downstream molecules (FIG. 13B and FIG. 13C).
(2) Further analysis of the results of the GEO database (GSE212301) showed a significant positive correlation between SP1 mRNA and E2F1 mRNA (FIG. 13D), suggesting that JP1 activates the Wnt pathway through the E2F1-SP1 axis, which then promotes the growth of dorsal hair in AGA mice.

The results of this example show that JP1 activates the Wnt pathway through the E2F1-SP1 axis.

### EXAMPLE 14

Previous studies have demonstrated that JP1 is a phosphorylation-modified small molecule peptide kinase that regulates downstream molecules and signaling pathways by promoting phosphorylation of interacting molecules. Literature reports that E2F1 is a downstream molecule of the MAPK signaling pathway, and when PKC/Raf/MEK/ERK signaling molecules in the MAPK signaling pathway are activated, the expression of E2F1 is also increased. Previous studies have found that JWA is involved in regulating MEK-ERK activity in the MAPK signaling pathway, but has no effect on upstream Raf.

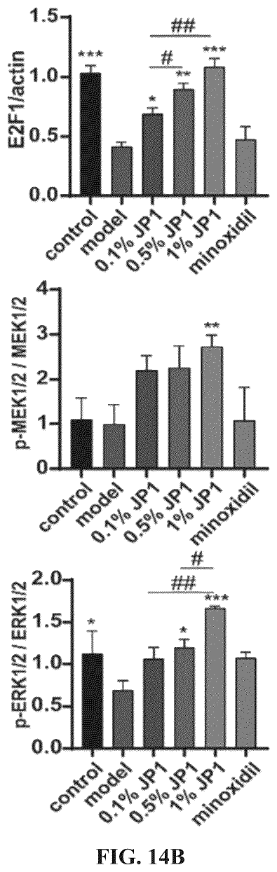

Accordingly, this example examined the protein levels of p-c-Raf, p-MEK, p-ERK, and E2F1 in the skin tissues of the model mice in Example 6, and the results showed that the expression levels of p-MEK and p-ERK in the MAPK signaling pathway were elevated under the intervention of JP1 (P<0.05, FIG. 14A), and there was a dose-effect relationship (FIG. 14B); however, there was no significant increase in the expression levels of p-c-Raf protein levels were not significantly altered. This suggests that JP1 is involved in activating the activities of p-MEK, p-ERK, but does not affect the activity of upstream Raf.

The results of this example suggest that JP1 acts through the MEK/ERK/E2F1 axis.

### EXAMPLE 15

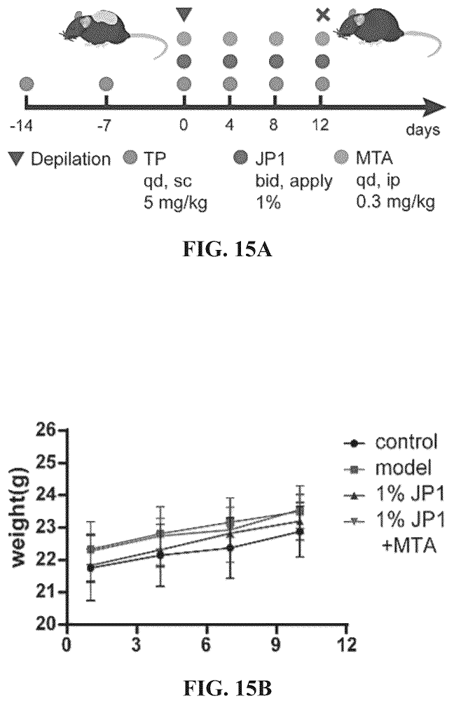

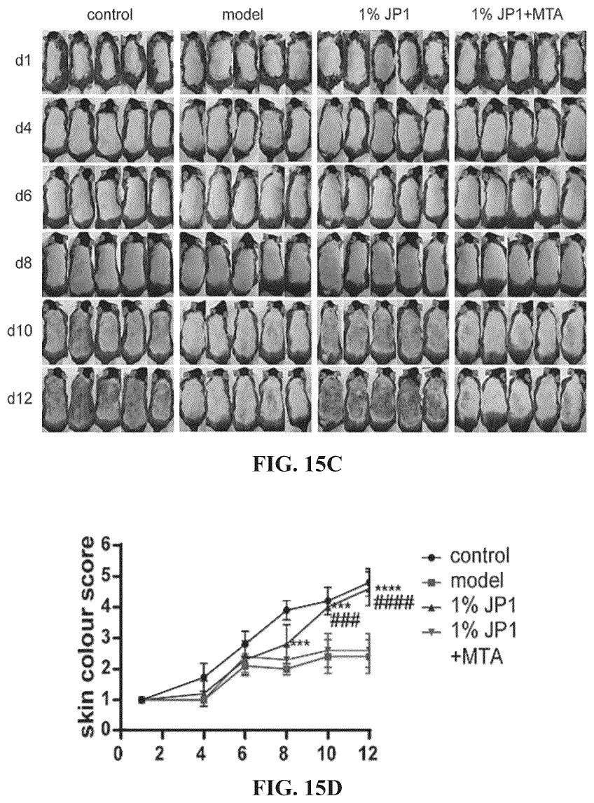

To verify that JP1 promotes AGA follicle regeneration through the activation of the Wnt/β-catenin signaling pathway via the MAPK/E2F1/SP1 signaling axis, the example constructed a mouse model of AGA with specific inhibition of SP1 (FIG. 15A). The specific experimental details are described according to “Animal model III” of “1. TP-induced AGA model” in “Experimental Methods”.

The body weights of the mice were measured every 2 days during the modeling period, and the results showed that there was no significant difference in the body weights of the mice in each group (P>0.05, FIG. 15B).

Analysis of the skin color on the back of mice in each group of the model showed that the skin of mice in the 1% JP1 group turned gray from day 8, while the skin of mice in the 1% JP1+MTA group had not yet changed; the skin of mice in the 1% JP1 group changed significantly to black-gray on day 10 (P<0.001); however, only a small portion of the area of the 1% JP1+MTA group underwent a color shift (FIG. 15C), and the above results suggested that the inhibition of SP1 by MTA blocked the hair follicle growth-promoting effect of JP1 on AGA model mice (FIG. 15D).

The results of this example show that inhibition of SP1 by MTA blocked the hair follicle growth promoting effect of JP1 on AGA model mice.

### EXAMPLE 16

To further confirm that JP1 promotes the growth of hair follicles on the back of AGA model mice through the SP1-regulated Wnt/β-catenin signaling pathway at the histomorphometric level, H&E staining was performed on the skin tissues of the model mice of Example 15 at different time points (days 4, 8, and 12 post-intervention).

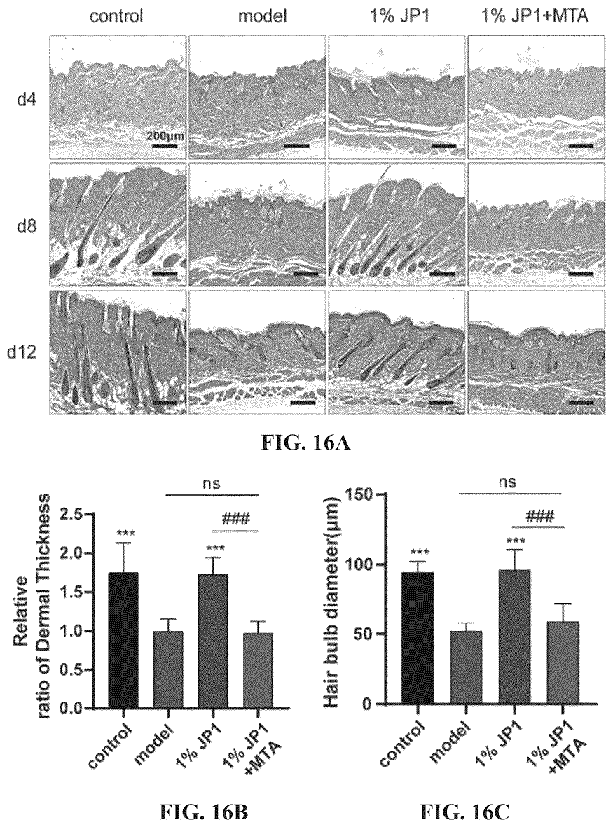

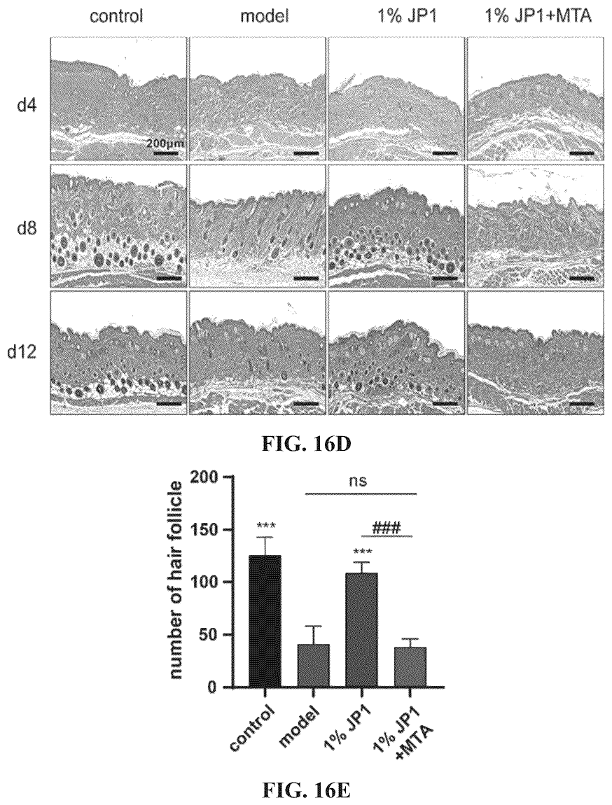

The results of longitudinal sectioning of skin tissues showed that on day 4, the morphology of hair follicles was consistent in all groups of mice; at day 8, hair follicles extended downward and the bulbous part of hairs expanded in the 1% JP1 group compared with the 1% JP1+MTA group, indicating that the hair follicles entered into the anagen phase earlier after the JP1 intervention (FIG. 16A). The 1% JP1 group increased the thickness of the dermis and the diameter of the bulb of hairs significantly (P<0.001), whereas the 1% JP1+MTA group and the model group had thinner dermis and smaller diameter of hair bulb (FIG. 16B and FIG. 16C).

The results of skin tissue transection showed that the total number of hair follicles was significantly more in the 1% JP1 group than in the model group and the 1% JP1+MTA group on both day 8 and day 12 (P<0.001, FIG. 16D and FIG. 16E).

The results of this example show that SP1 inhibition by MTA blocked the histomorphometric changes of JP1 promoting hair follicle growth in AGA model mice.

### EXAMPLE 17

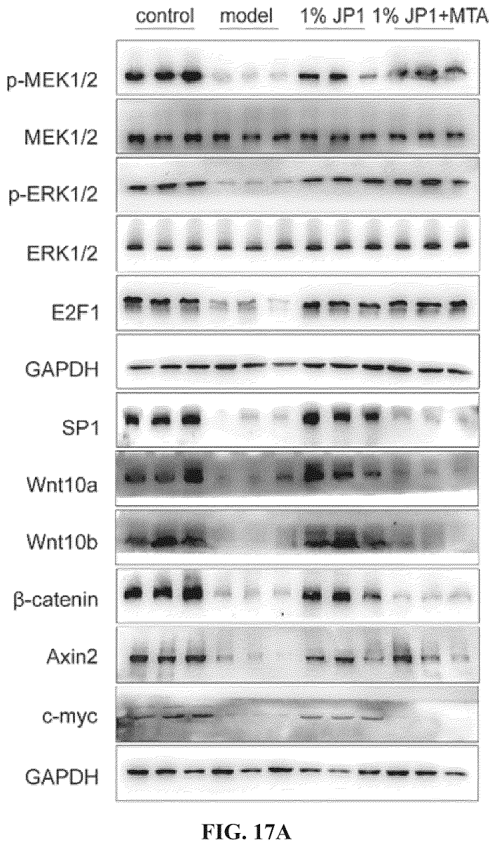

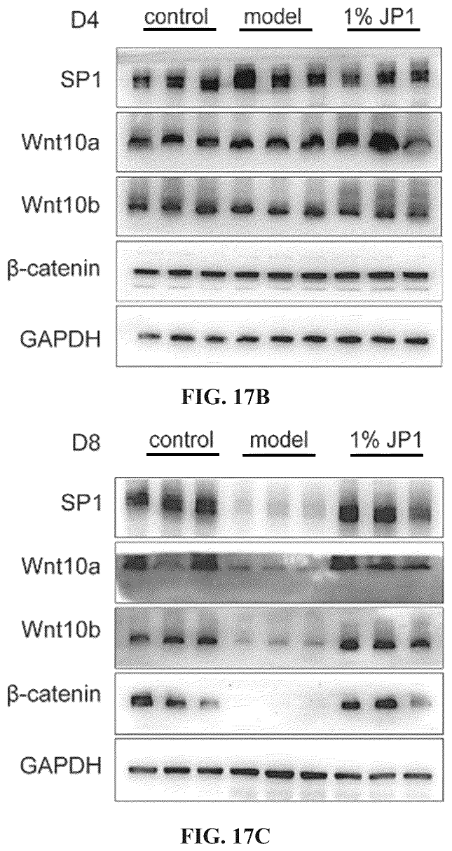

In this example, relevant molecular immunoblotting assays were performed on the dorsal skin tissues of the mice in each group of the AGA model in Example 15 above, and the results showed that the inhibition of SP1 had no significant effect on the expression levels of the upstream molecules MEK/ERK/E2F1 of SP1, while the expression levels of both SP1 and its downstream molecules were inhibited, thereby blocking the growth-promoting effect of JP1 on the hair follicle (FIG. 17A).

The results of the dorsal skin appearance map of Example 15 showed that differences in skin color began to appear between the groups at day 8 (FIG. 15C). To verify the change rules of key molecules of the pro-follicular growth signaling pathway regulated by JP1 at different time points in the model, the immunoblotting results of this example found that the expression levels of the above key molecules did not change significantly in the control group, the model group and the 1% JP1 group on day 4 of the intervention; while the protein expression levels of four molecules in the 1% JP1 group increased significantly on day 8, consistent with the change in skin color (FIG. 17A and FIG. 17B).

The results of this example show that MTA can block the effect of JP1 on the expression of SP1 downstream molecules.

### EXAMPLE 18

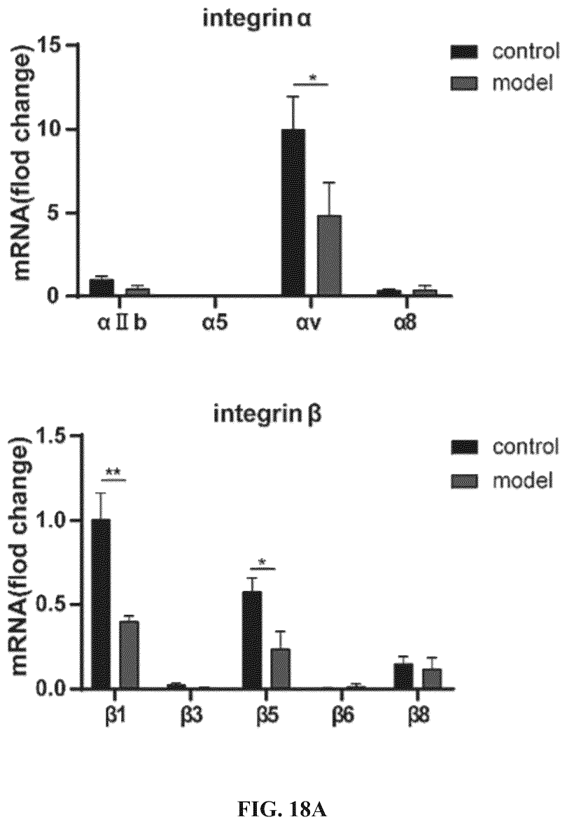

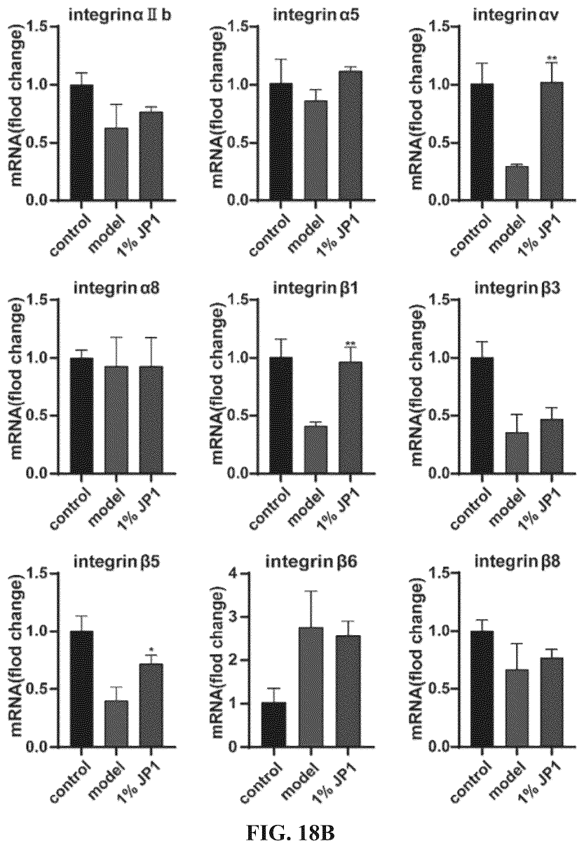

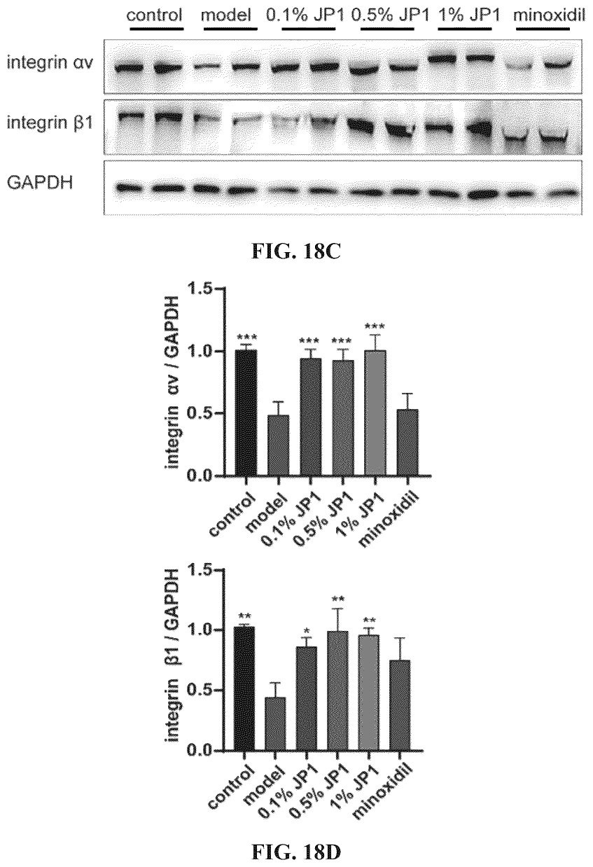

On the basis of knowing the phenotypic and molecular mechanisms by which JP1-targeting peptide effectively promotes the growth of hair follicles in the skin of AGA model mice, the example used the skin tissue of the model mice in Example 6 to further examine the dynamic process of penetration through the skin and the exact molecule targeting to the hair follicle of JP1 active peptide labeled with fluorescein FITC and connected to the targeting integrin RGD. It is known that the RGD target head can be targeted to bind αIIb, αv, α5, α8 of the integrin α isoforms and β1, β3, β5, β6 and β8 of the integrin β isoforms. The results of this example on the mRNA expression levels of integrin subunits in skin tissues that can be bound by the RGD showed that both the normal control group and TP induced AGA mice showed the highest expression of αv in the integrin α subtype of the skin tissue; β1 expression was significantly highest among integrin β subtypes (FIG. 18A). It is suggested that in the skin tissues of AGA model mice, JP1 targets hair follicle tissues mainly through integrin αvβ1.

In this example, the mRNA expression levels of RGD-targeting molecules in the skin tissues of three groups of mice, namely, the AGA model control group, the model group and the JP1-treated group, were further examined. The results showed that the expression levels of integrins αv and β1 were significantly decreased in the skin tissues of mice in the TP model group, whereas the expression of integrins αv and β1 was significantly elevated after JP1 intervention (P<0.01), followed by β5. At the end of the model, integrin αv and β1 expression in the JP1 intervention group had returned to control levels (P<0.01, FIG. 18B).

The protein levels of integrin αv and β1 were further verified by immunoblotting in this example, and the results similarly showed that in JP1 reversed the TP-down-regulated integrin αv and β1 expression levels (P<0.01, FIG. 18C and FIG. 18D); furthermore, although the above series of model results show that minoxidil has a similar promoting effect on hair follicle growth as JP1, it does not have a significant effect on the expression levels of integrin αv and β1 inhibited by TP (FIGS. 18C-18D).

The results of this example show that JP1 primarily targets integrin αvβ1 into hair follicle tissue.

### EXAMPLE 19

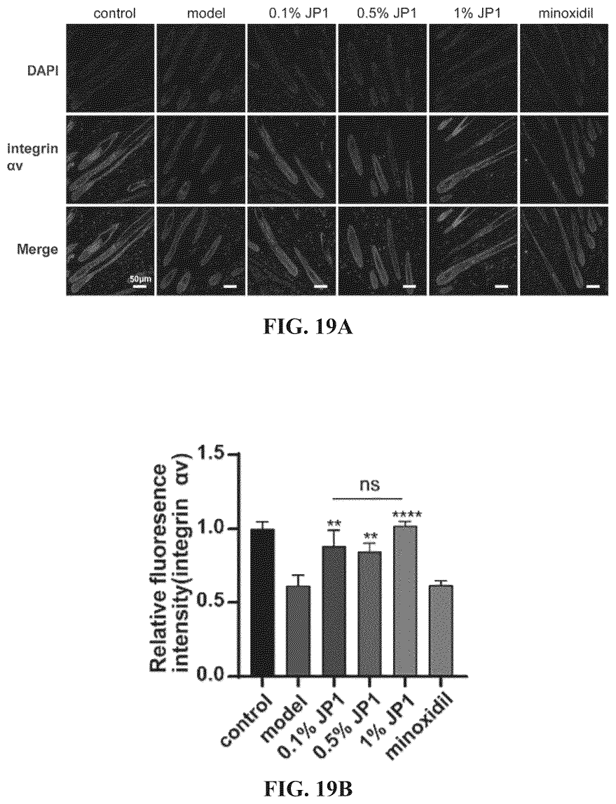

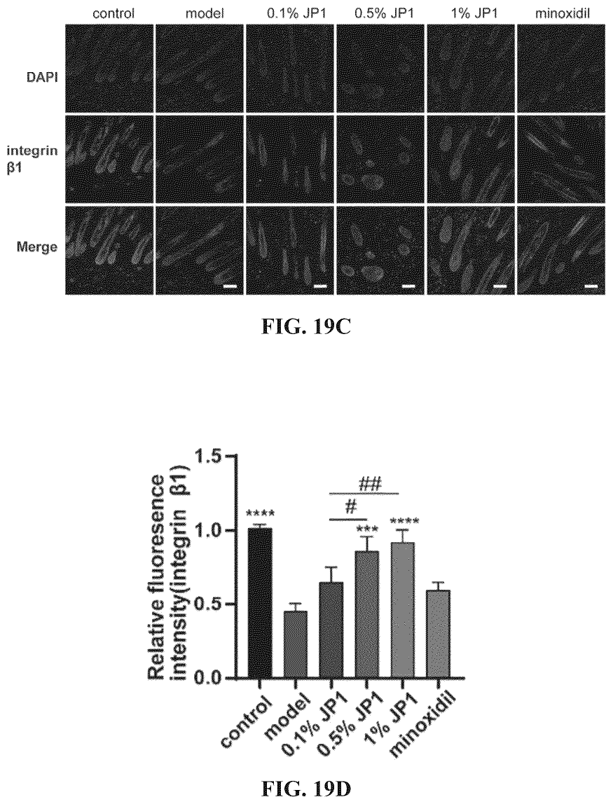

To verify whether JP1 targeted integrin molecules are localized in hair follicles in the skin tissue, this example detected the expression levels, tissue localization, and distribution of integrin αv and integrin β1 in the skin tissue of the mouse model in Example 6 using immunofluorescence staining. The results showed that the expression of integrin αv (FIGS. 19A-19B) and integrin β1 (FIGS. 19C-19D) was elevated in each JP1 intervention group, which were localized in the site of hair follicles, and the changes in the expression of the target molecules in each group were consistent with the protein expression levels detected by immunoblotting.

The results of this example show that JP1 not only targets integrin αvβ1 into the hair follicle cells in the AGA model, but also reverses the integrin αvβ1 expression level in the hair follicle cells inhibited by TP.

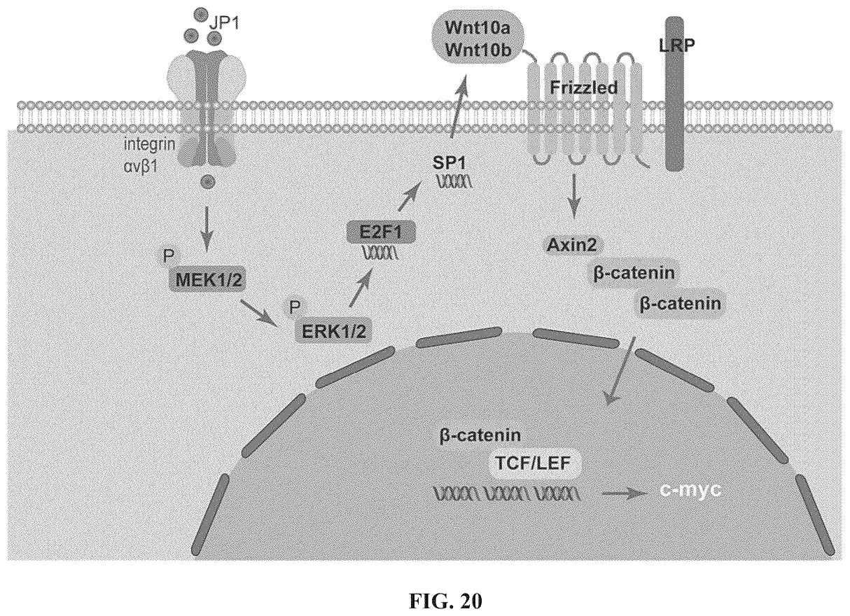

Combining the results of the above examples, it can be seen that, as shown in FIG. 20, the molecular mechanism by which JP1 promotes the growth of hair follicles in the AGA model is as follows:
(1) JP1, as a functional peptide fragment of JWA gene, enters into hair follicle target cells through RGD targeting integrin αvβ1 after topical skin coating, up-regulates the expression of the integrin αvβ1, which in turn increases the content of JP1 peptide in the hair follicle target cells and promotes the hair follicle growth with a positive feedback effect.
(2) JP1 activates the MEK/ERK/E2F1 signaling axis, thereby promoting the expression of SP1 and activating Wnt10a/Wnt10b, binds to the Frizzled receptor family and LRP5/LRP6 co receptors, increasing the aggregation of free β-catenin into the nucleus, and binds to the LEF/TCF transcription complex, activates downstream signals such as c-myc, and promotes the growth of AGA hair follicles.

### EXAMPLE 20

This example is to verify the anti-AGA effect of various JWA peptides except JP1.

This example uses the JWA peptides shown in the table below (JP2-JP39, note: Acp is the abbreviation symbol for 6-aminocaproic acid) for detection according to Examples 6 to 12. The amino acid serine(S) of each JWA peptide is phosphorylated.

| No. | Sequence | No. | Sequence | No. | Sequence |
|---|---|---|---|---|---|
| JP2 | FPGSDRF-G-RGD | JP3 | FPGSDRF-(G)₄- | JP4 | FPGSDRF-(G)₁₀- |
| | (SEQ ID NO: 5) | | RGD | | RGD |
| | | | (SEQ ID NO: 6) | | (SEQ ID NO: 7) |
| JP5 | FPGSDRF-A-RGD | JP6 | FPGSDRF-A-G- | JP7 | FPGSDRF-A-(G)₄- |
| | (SEQ ID NO: 8) | | RGD | | RGD |
| | | | (SEQ ID NO: 9) | | (SEQ ID NO: 10) |
| JP8 | FPGSDRF-A-(G)₁₀- | JP9 | FFPGSDRF-RGD | JP10 | FFPGSDRF-G-RGD |
| | RGD | | (SEQ ID NO: 12) | | (SEQ ID NO: 13) |
| | (SEQ ID NO: 11) | | | | |
| JP11 | FFPGSDRF-(G)₄-R | JP12 | FFPGSDRF-(G)₁₀- | JP13 | FFPGSDRF-A-RGD |
| | GD | | RGD | | (SEQ ID NO: 16) |
| | (SEQ ID NO: 14) | | (SEQ ID NO: 15) | | |
| JP14 | FFPGSDRF-A-G- | JP15 | FFPGSDRF-A-(G)₁₀- | JP16 | (R)₉-FPGSDRF- |
| | RGD | | RGD | | RGD |
| | (SEQ ID NO: 17) | | (SEQ ID NO: 18) | | (SEQ ID NO: 19) |
| JP17 | (R)₉-FPGSDRF- | JP18 | (R)₉-FPGSDRF-A- | JP19 | (R)₉-FPGSDRF-A- |
| | (G)₁₀-RGD | | RGD | | (G)₁₀-RGD |
| | (SEQ ID NO: 20) | | (SEQ ID NO: 21) | | (SEQ ID NO: 22) |
| JP20 | (R)₉-F-FPGSDRF- | JP21 | (R)₉-F-FPGSDRF- | JP22 | (R)₉-F-FPGSDRF- |
| | RGD | | (G)₁₀-RGD | | A-RGD |
| | (SEQ ID NO: 23) | | (SEQ ID NO: 24) | | (SEQ ID NO: 25) |
| JP23 | (R)₉-F-FPGSDRF- | JP24 | 6-aminocaproic | JP25 | 6-aminocaproic |
| | A-(G)₁₀-RGD | | acid-FPGSDRF- | | acid-FPGSDRF- |
| | (SEQ ID NO: 26) | | RGD | | (G)₁₀-RGD |
| | | | (SEQ ID NO: 27) | | (SEQ ID NO: 7) |
| JP26 | 6-aminocaproic | JP27 | 6-aminocaproic | JP28 | 6-aminocaproic |
| | acid-FPGSDRF- | | acid-FPGSDRF-A- | | acid-F-FPGSDRF- |
| | A-RGD | | (G)₁₀-RGD | | RGD |
| | (SEQ ID NO: 8) | | (SEQ ID NO: 11) | | (SEQ ID NO: 12) |
| JP29 | 6-aminocaproic | JP30 | 6-aminocaproic | JP31 | 6-aminocaproic |
| | acid-F-FPGSDRF- | | acid-F-FPGSDRF- | | acid-F-FPGSDRF- |
| | (G)₁₀-RGD | | A-RGD | | A-(G)₁₀-RGD |
| | (SEQ ID NO: 15) | | (SEQ ID NO: 16) | | (SEQ ID NO: 18) |
| JP32 | 6-aminocaproic | JP33 | 6-aminocaproic | JP34 | 6-aminocaproic |
| | acid-(R)₉- | | acid-(R)₉- | | acid-(R)₉- |
| | FPGSDRF-RGD | | FPGSDRF-(G)₁₀- | | FPGSDRF-A-RGD |
| | (SEQ ID NO: 19) | | RGD | | (SEQ ID NO: 21) |
| | | | (SEQ ID NO: 20) | | |
| JP35 | 6-aminocaproic | JP36 | 6-aminocaproic | JP37 | 6-aminocaproic |
| | acid-(R)₉- | | acid-(R)₉-F- | | acid-(R)₉-F- |
| | FPGSDRF-A-(G)₁₀- | | FPGSDRF-RGD | | FPGSDRF-(G)₁₀- |
| | RGD | | (SEQ ID NO: 23) | | RGD |
| | (SEQ ID NO: 22) | | | | (SEQ ID NO: 24) |
| JP38 | 6-aminocaproic | JP39 | 6-aminocaproic | | |
| | acid-(R)₉-F- | | acid-(R)₉-F- | | |
| | FPGSDRF-A-RGD | | FPGSDRF-A- | | |
| | (SEQ ID NO: 25) | | (G)₁₀-RGD | | |
| | | | (SEQ ID NO: 26) | | |

Due to space limitations, specific experimental data are not listed in the example. The obtained experimental data indicate that the test results of each JWA peptide according to Examples 6 to 15 are basically consistent with that of JP1.

### Conclusion

Combining the results of the studies of the above examples, the disclosure successfully constructed a TP-induced androgenetic alopecia mouse model, either by intraperitoneal injection or topical skin-coating interventions, and the series of JWA peptides represented by JP1 dose-dependently promoted the growth of dorsal hairs in the TP-induced AGA mouse model. Mechanistically, the above JWA peptides promoted the expression of nuclear transcription factor SP1 by activating the p-MEK/p-ERK/E2F1 axis, which in turn activated the Wnt pathway. Wnt10a and Wnt10b bound to the receptors on the cell membrane, which led to the aggregation of a large amount of free β-catenin into the nucleus and activation of downstream signaling, thus achieving the regulation of the follicle cycle and the promotion of hair growth. In addition, the above JWA polypeptide improved the ecological microenvironment of the hair follicle by targeting integrin αvβ1 into the hair follicle target cells and upregulating the expression level of integrin αvβ1 inhibited by TP. The disclosure not only provides scientific evidence for the aforesaid JWA peptide for the treatment of AGA, but also elucidates a new molecular mechanism for how the aforesaid JWA peptide activates hair follicle stem cells for hair growth.

The materials, methods, experimental model conditions, etc. used in the above examples are shown below.

### 1. Experimental Peptides

The series of JWA peptides represented by JP1 were synthesized by GL Biochem (Shanghai) Ltd. and Hybio Pharmaceutical Co. Ltd. Purity >98%, water-soluble. FITC-JP1 was synthesized by Zhejiang Paiti Biotechnology Co., Ltd. (Hangzhou, China) and its purity was confirmed to be >98% by high-performance liquid chromatography. JWA peptide freeze-dried powder can be stored for a long time at −20° C.

### 2. Reagents and Kits Used in the Experiments

Specific information on the reagents used in the disclosure is shown in Table 1.

TABLE 1
Specific reagents used in the disclosure

| Reagent Name | Reagent Manufacturer |
|---|---|
| Glycine, Tris, SDS, NaCl, | Shanghai Sinopharm Group Chemical |
| | Reagent Co. |
| RIPA lysate | Suzhou New Saimei Co. |
| BCA Protein Concentration Measurement Kit | Shanghai Biyuntian Biotechnology Co. |
| Protease inhibitor mix (100×) | Shanghai Biyuntian Biotechnology Co. |
| Phosphatase inhibitor mix (50×) | Shanghai Biyuntian Biotechnology Co. |
| PMSF | Shanghai Biyuntian Biotechnology Co. |
| Tween 20 | Nanjing Chemical Reagent Co. |
| 30% Acrylamide | Wuhan Xavier Biotechnology Co. |
| 10% SDS | Shanghai Biyuntian Biotechnology Co. |
| AP | Shanghai Biyuntian Biotechnology Co. |
| TEMED | Sigma, USA |
| Skimmed milk powder | Maxigenes Dairy Australia Pty Ltd |
| Methanol | Sinopharm Chemical Reagent Co. |
| Anhydrous ethanol | Sinopharm Chemical Reagent Co. |
| Primary antibody diluent | Shanghai Biyuntian Biotechnology Co. |
| (western/immunofluorescence) | |
| Disodium hydrogen phosphate | Xilong Science Co. |
| Sodium bicarbonate | Xilong Science Co. |
| 180 kDa Prestained Protein Marker | Nanjing Novozymes |
| Normal ECL Chemiluminescent Assay Kit | Nanjing Novozymes Co. |
| Sensitized ECL Chemiluminescent Assay Kit | Nanjing Novozymes Co. |
| RNAeasyTM Animal RNA Extraction Kit | Shanghai Biyuntian Biotechnology Co. |
| (Centrifugal Column) | |
| 4% paraformaldehyde fixative | Wuhan Xavier Biotechnology Co. |
| HiScript III RT SuperMix for qPCR (+gDNA | Novozymes Nanjing |
| wiper) Reverse Transcription Kit | |
| AceQ qPCR SYBR Green Master Mix | Novozymes Nanjing |
| Fluorescent Quantitative PCR Assay Kit | |
| 20 × Antigen Repair Solution | Wuhan Xavier Biotechnology Co. |
| Anti-fluorescent bursting agent containing DAPI | Wuhan Xavier Biotechnology Co. |

### 3. Antibodies Used in the Experiments

Specific information on the antibodies used in the disclosure is given in Table 2.

TABLE 2
Specific antibodies used in the disclosure

| | | Catalog | Dilution |
|---|---|---|---|
| Antibody Name | Manufacturer | Number | Ratio |
| JWA | Self-prepared | — | 1:100 |
| β-catenin | Proteintech | 51067-1-AP | 1:200 |
| c-myc | Proteintech | 10828-1-AP | 1:400 |
| Wnt10a | Proteintech | 26238-1-AP | 1:1000 |
| Wnt10b | Proteintech | 67210-1-AP | 1:5000 |
| SP1 | Proteintech | 21962-1-AP | 1:500 |
| E2F1 | Cell Signaling | 3742 | 1:1000 |
| | Technology | | |
| p-MEK1/2 | Cell Signaling | 2338s | 1:1000 |
| | Technology | | |
| MEK1/2 | Cell Signaling | 9122s | 1:1000 |
| | Technology | | |
| p-ERK1/2 | Affinity Bioscience | AF1015 | 1:2000 |
| | LTD | | |
| ERK1/2 | Affinity Bioscience | BF8004 | 1:2000 |
| | LTD | | |
| Axin2 | Proteintech | 20540-1-AP | 1:1500 |
| GAPDH | Beyotime | AF0006 | 1:1000 |
| Actin | Beyotime | AA128 | 1:1000 |
| integrin αν | Abcam | ab179475 | 1:500 |
| integrin β1 | Proteintech | 26918-1-AP | 1:1000 |
| HRP labeled goat | Beyotime | A0208 | 1:1000 |
| anti mouse | | | |
| HRP labeled goat | Beyotime | A0216 | 1:1000 |
| anti rabbit | | | |
| Alexa Fluor ® 555 | Abcam | ab150078 | 1:500 |
| Alexa Fluor ® 488 | Abcam | ab150113 | 1:500 |

### 4. Primer Synthesis

The primers used in this disclosure were commissioned to be synthesized by Shanghai Jierui Bioengineering Company Limited, and their specific sequences are shown in Table 3.

TABLE 3
Specific sequences of primers used in this study

| Primers | Sequences (5′-3′) |
|---|---|
| mouse AR F | CCTTGGATGGAGAACTACTCCG (SEQ ID NO: 29) |
| mouse AR R | TCCGTAGTGACAGCCAGAAGCT (SEQ ID NO: 30) |
| mouse SRD5A2 F | CATCCACAGTGACTGCATGCTG (SEQ ID NO: 31) |
| mouse SRD5A2 R | AAGGCTGGAACAGACCAAGTGG (SEQ ID NO: 32) |
| mouse GAPDH F | CATCACTGCCACCCAGAAGACTG (SEQ ID NO: 33) |
| mouse GAPDH R | ATGCCAGTGAGCTTCCCGTTCAG (SEQ ID NO: 34) |
| mouse IGF-1 F | GTGGATGCTCTTCAGTTCGTGTG (SEQ ID NO: 35) |
| mouse IGF-1 R | TCCAGTCTCCTCAGATCACAGC (SEQ ID NO: 36) |
| mouse HGF F | GTCCTGAAGGCTCAGACTTGGT (SEQ ID NO: 37) |
| mouse HGF R | CCAGCCGTAAATACTGCAAGTGG (SEQ ID NO: 38) |
| mouse CTNNB1 F | GTTCGCCTTCATTATGGACTGCC (SEQ ID NO: 39) |
| mouse CTNNB1 R | ATAGCACCCTGTTCCCGCAAAG (SEQ ID NO: 40) |
| mouse LEF1 F | ACTGTCAGGCGACACTTCCATG (SEQ ID NO: 41) |
| mouse LEF1 R | GTGCTCCTGTTTGACCTGAGGT (SEQ ID NO: 42) |
| mouse wnt1 F | CGAGAGTGCAAATGGCAATTCCG (SEQ ID NO: 43) |
| mouse wnt1 R | GATGAACGCTGTTTCTCGGCAG (SEQ ID NO: 44) |
| mouse wnt3a F | AACTGCACCACCGTCAGCAACA (SEQ ID NO: 45) |
| mouse wnt3a R | AGCGTGTCACTGCGAAAGCTAC (SEQ ID NO: 46) |
| mouse wnt7b F | TTCTCGTCGCTTTGTGGATGCC (SEQ ID NO: 47) |
| mouse wnt7b R | CACCGTGACACTTACATTCCAGC (SEQ ID NO: 48) |
| mouse wnt10a F | GCTCCTGTTCTTCCTACTGCTG (SEQ ID NO: 49) |
| mouse wnt10a R | ATGTCAGGCACACTGTGTTGGC (SEQ ID NO: 50) |
| mouse wnt10b F | ACCACGACATGGACTTCGGAGA (SEQ ID NO: 51) |
| mouse wnt10b R | CCGCTTCAGGTTTTCCGTTACC (SEQ ID NO: 52) |
| mouse SP1 F | CTCCAGACCATTAACCTCAGTGC (SEQ ID NO: 53) |
| mouse SP1 R | CACCACCAGATCCATGAAGACC (SEQ ID NO: 54) |
| mouse MYC F | TCGCTGCTGTCCTCCGAGTCC (SEQ ID NO: 55) |
| mouse MYC R | GGTTTGCCTCTTCTCCACAGAC (SEQ ID NO: 56) |
| mouse E2F1 F | GGATCTGGAGACTGACCATCAG (SEQ ID NO: 57) |
| mouse E2F1 R | GGTTTCATAGCGTGACTTCTCCC (SEQ ID NO: 58) |
| mouse FOXD3 F | CAAGAACAGCCTGGTGAAGCCA (SEQ ID NO: 59) |
| mouse FOXD3 R | ACGGTTGCTGATGAACTCGCAG (SEQ ID NO: 60) |
| mouse FOXI1 F | GTTCGCCTTCATTATGGACTGCC (SEQ ID NO: 61) |
| mouse FOXI1 R | AACTCCATCCGCCACAACCTGT (SEQ ID NO: 62) |
| mouse integrin αIIb F | TTCTTGGGTCCTAGTGCTGTT (SEQ ID NO: 63) |
| mouse integrin αIIb R | CGCTTCCATGTTTGTCCTTATGA (SEQ ID NO: 64) |
| mouse integrin αv F | CCGTGGACTTCTTCGAGCC (SEQ ID NO: 65) |
| mouse integrin αv R | CTGTTGAATCAAACTCAATGGGC (SEQ ID NO: 66) |
| mouse integrin α5 F | CTTCTCCGTGGAGTTTTACCG (SEQ ID NO: 67) |
| mouse integrin α5 R | GCTGTCAAATTGAATGGTGGTG (SEQ ID NO: 68) |
| mouse integrin α8 F | CGAAGCCGAACTCTTTGTTATCA (SEQ ID NO: 69) |
| mouse integrin α8 R | GGCCTCAGTCCCTTGTTGT (SEQ ID NO: 70) |
| mouse integrin β1 F | ATGCCAAATCTTGCGGAGAAT (SEQ ID NO: 71) |
| mouse integrin β1 R | TTTGCTGCGATTGGTGACATT (SEQ ID NO: 72) |
| mouse integrin β3 F | CCACACGAGGCGTGAACTC (SEQ ID NO: 73) |
| mouse integrin β3 R | CTTCAGGTTACATCGGGGTGA (SEQ ID NO: 74) |
| mouse integrin β5 F | GAAGTGCCACCTCGTGTGAA (SEQ ID NO: 75) |
| mouse integrin β5 R | GGACCGTGGATTGCCAAAGT (SEQ ID NO: 76) |
| mouse integrin β6 F | CAACTATCGGCCAACTCATTGA (SEQ ID NO: 77) |
| mouse integrin β6 R | GCAGTTCTTCATAAGCGGAGAT (SEQ ID NO: 78) |
| mouse integrin β8 F | AGTGAACACAATAGATGTGGCTC (SEQ ID NO: 79) |
| mouse integrin β8 R | TTCCTGATCCACCTGAAACAAAA (SEQ ID NO: 80) |

### 5. Self-Formulated Reagents

1) PBS solution: 3.5 g of Na₂HPO₄-12H₂O, 8 g of NaCl, 0.2 g of KCl and 0.2 g of Na₂HPO₄ were mixed and double-distilled water was added to a total volume of 1000 mL, and store at 4° C. after autoclaving.

2) RIPA lysate: 5 g of sodium deoxycholate, 0.5 g of SDS and 4.39 g of NaCl were dissolved in 25 mL of 1 M Tris-HCl (pH=7.6) and 5 mL of Triton X-100, and double-distilled water was added to a total volume of 500 mL, and store at 4° C.

3) 5× protein sampling buffer (5×SDS): 0.5 g of SDS and 25 mg of bromophenol blue were mixed, and 5 mL of glycerol and 5 mL of 1 M Tris-HCl (pH=6.8) buffer added; the solution was subpackaged in 1 mL each and stored at −20° C.; 50 μL of β-me (mercaptoethanol) to each one before use.

4) Concentrated gel (upper layer of gel) buffer: 121.1 g of Tris powder was dissolved in double-distilled water, and concentrated hydrochloric acid was added to adjust pH=6.8, and finally double-distilled water was added to a total volume of 1000 mL.

5) Separation gel (lower gel) buffer: 181.5 g of Tris powder was dissolved in double-distilled water, and concentrated hydrochloric acid was added to adjust pH=8.8, and finally double-distilled water was added to a total volume of 1000 mL.

6) 30% acrylamide solution: 290 g of acrylamide and 10 g of methylene bifurcated acrylamide were dissolve in double-distilled water and make a constant volume of 1000 mL, stored at 4° C. away from light.

7) 1× Electrophoresis buffer: 3.03 g of Tris, 14.4 g of glycine, and 1 g of SDS were dissolved in double-distilled water in a beaker, and the volume was constant to 1000 mL.

8) 1× transfiltration buffer: 2.4 g of Tris, 11.5 g of glycine, and 200 mL of methanol were mixed, and then double-distilled water was added to a volume of 1000 mL.

9) 1×TBST buffer: 24.2 g of Tris and 80 g of NaCl were added to a beaker, double-distilled water was added, and the pH was adjusted to 7.6 with concentrated hydrochloric acid; double-distilled water was added to the breaker to 1,000 mL and stored at room temperature, i.e., 10×TBS was obtained. 100 mL of 10×TBS was added 900 mL of double-distilled water, 1 mL of Tween-20 was further added and mixed for use.

10) Closure solution (5% skimmed milk powder): 10 g of skimmed milk powder and 200 mL 1×TBST were completely mixed.

11) Elution solution: 15 g of glycine and 1 g of SDS were dissolved in double-distilled water, and then 10 mL of Tween-20 was added, the pH was adjusted to 2.2 with concentrated hydrochloric acid, and the volume was constant to 1000 mL with double-distilled water.

12) JP1 intraperitoneal injection solvent: saline

13) JP1 skin-coating solvent: 10% saline+40% anhydrous ethanol+50% propylene glycol; 50 μL/per animal/dose.

14) Modeling drug: Testosterone propionate (TP, prepared by isodilution method), 25 mg/mL of testosterone propionate injection was diluted to 2.5 mg/ml using soybean oil, and fully shaken for 2-3 min, 5 mg/kg d, 50 μL/per animal, 1 time/day. Testosterone propionate (TP, 25 mg/kg) was purchased from Sichuan Jinke Pharmaceutical Co. Ltd.

15) 5% Minoxidil: purchased from Zhejiang Wansheng Pharmaceutical Co., Ltd, topical application of skin, 100 μL/per animal, once/day.

16) Mithramycin A (MTA): SP1 inhibitor, purchased from MCE, solvent: 10% DMSO+40% PEG300+5% Tween80+45% saline.

### Experimental Methods

### 1. TP Induced AGA Model

The mice used in this study were 5-week-old C57BL/6J male mice (18-20 g), which were purchased from Jiangsu Jicui Pharmachem Biotechnology Co. All the animals used in the experiments were housed in the SPF-level environment of the Medical Laboratory Animal Center of Nanjing Medical University, and the relevant animal experimental operations were approved by the Animal Ethics Committee of Nanjing Medical University. The control group was left untreated, and the other groups were injected with TP subcutaneously on their backs every day. Two weeks later, the hair on the backs of the mice was shaved with an electric razor, covering an area of 2 cm×4 cm, and then the residual hair was removed with a hair removal cream. The subcutaneous injections of TP on the shaved backs of the mice in the AGA model was continued until the end of the experimental period, and the administration of TP in each of the treatment groups was begun on the 1st day of the dehairing process.

### Animal Model I

In addition to the normal control group (denoted as control) n=3, all mice injected subcutaneously with TP were randomly divided into the following groups (n=3): the model group (denoted as model), the 1 wt. % JP1 and 2% JP1 (intraperitoneal), the 1% JP1 and 2% JP1 (topical skin-coating, denoted as apply), the 5% minoxidil group, and the 2% JP1 (intraperitoneal) combined with 5% minoxidil group. The mice were administered continuously for 35 days, euthanized.

### Animal Model II

In addition to the normal control group (denoted as control) n=20, all mice injected subcutaneously with TP were randomly divided into the following groups (n=20): the model group (denoted as model), the 0.1% JP1, the 0.5% JP1, the 1% JP1 (all administered by topical dermal application), and the 5% minoxidil group. Mice were observed for changes in dorsal skin color during the experiment, photographed with a camera at key time points, and euthanized in four batches (n=5 per batch) at key time points.

### Animal Model III

In addition to the normal control group (denoted as control) n=20, all mice injected subcutaneously with TP were randomly divided into the following groups (n=20): the model group (denoted as model), the 1% JP1 (skin-coated), and the 1% JP1+MTA group. Mice were observed for dorsal skin color changes during the experiment, photographed with a camera at key time points, and euthanized in batches at key time points (n=5 in each group at a time). MTA was administered at a dose of 0.3 mg/kg/d, qd, intraperitoneally, 100 μL/dose.

Mice were weighed every two days during the experiment. Skin color was assessed against skin score charts. The mice were euthanized and treated at the end of the experiment, and skin tissues were taken from the dorsal decorticated area of the mice. Some of the tissues were fixed with 4% paraformaldehyde solution and then paraffin sections were made for H&E or immunofluorescence staining, etc., and some of the tissues were stored in the refrigerator at −80° C. for relevant molecular expression assays (qRT-PCR, Western Blot, IP, etc.).

### 2. JP1 Stability Experiment

The stability of JP1 dissolved in a skin-coating solvent was tested at different time points. JP1 was prepared with the skin-coating solvent, and a solvent control group and a 10 mM JP1 group were set up. The prepared solution was stored in a refrigerator at 4° C. The five time points of 0, 1, 3, 7 and 14 days were detected by high performance liquid chromatography (HPLC), and the standard curve was plotted with the peak area as the vertical coordinate and the concentration of JP1 as the horizontal coordinate.

### 3. JP1 Permeability Experiment

15 C57BL/6J male mice were dorsally depilated after two weeks of continuous TP injection. A 1% FITC-JP1 solution was applied locally to the depilated area of the mice on the 1st day after depilation, no light, in five groups of 0.5 h, 1 h, 2 h, 4 h and 8 h (n=3).

The back skin was removed at the corresponding time points and fixed in 4% paraformaldehyde. After 48 h of fixation, the skin tissue was successively dehydrated in 20% and 30% sucrose solution for 2 days each. After gradient dehydration, sections (20 μm) were made using a frozen microtome. Zeiss inverted fluorescence microscope was used to photograph the fluorescent sections.

### 4. Protein Extraction and Concentration Determination

4.1) Protein extraction: 70 mg of skin tissue was placed in a 1.5 mL centrifuge tube, and 500 μL of tissue lysate RIPA containing protease inhibitor and phosphatase inhibitor was added. The tissue was placed on an ice box, pulverized by a tissue grinder, and then ultrasonically crushed to obtain a tissue suspension, which was then placed in a rotary windmill at 4° C. to be lysed for 2 h. The centrifuge was set at 12,000×g, 4° C., centrifuged for 15 min, and the supernatant was collected as the total protein.

4.2) Determination of Protein Concentration with BCA Kit

A) Preparation of standard curve: 10 μL of BSA standard protein at a concentration of 5 mg/mL was added to 90 μL of saline to formulate BSA standard protein having a final concentration of 0.5 mg/mL. 0, 1, 2, 4, 8, 12, 16, 20 μL of the standard protein was added to each well in a 96-well plate, and then each well was controlled to 20 μL with saline.

B) Preparation of protein samples: 1 μL of protein sample was added to per well and then 19 μL of saline was added.

C) Preparation of BCA working solution: a working solution was prepared according to the ratio of solution A: solution B=50:1, and 200 μL of the working solution was added to each well, and incubated at 37° C. for 30 min, protected from light.

D) Determination of protein concentration: after 30 min, the absorbance of the enzyme marker was set to 572 nm, and the concentration of the protein sample was calculated according to the standard curve of protein concentration.

### 5. Western Blotting

5.1) Heating of protein samples: the total protein content of each tissue was 50 μg; calculate the volume of a corresponding protein sample, make up the volume with saline to 16 μL, then add 4 μL of 5×SDS sampling buffer, heat at 100° C. for 5 min, and then cool at room temperature and store at −20° C.

5.2) Preparation of SDS-PAGE gel: firstly, clean the assembled glass plate and check the leakage, and prepare an appropriate concentration of a lower separation gel according to the molecular weight of the required detection protein. Choose low concentration separation gel for large molecular weight and higher concentration separation gel for small molecular weight, usually 10% separation gel is used. To a centrifuge tube, the double-distilled water, 30% acrylamide solution, lower gel buffer, 10% SDS, 10% AP, and TEMED were added, mixed, and then filled in the gap between the glass plates. Anhydrous ethanol was added to prevent the lower gel from drying out, and then the lower gel was solidified for about 40 min at room temperature. The anhydrous ethanol was dried. Then, the upper layer of glue was prepared, and a 1.0 mm sample comb was inserted into the upper layer of glue to avoid air bubbles, and solidified for about 30 min.

5.3) Protein sampling: fix the gel on the electrophoresis rack, fill the electrophoresis tank with electrophoresis solution, pull out the sample comb in the upper layer of the gel, add 10 μL of protein samples to each well with a pipette gun with a 10 μL volume range, and finally add 2 μL of molecular weight marker (marker).

5.4) Electrophoresis: electrophoresis was carried out under constant voltage, 80 V for about 30 min, and the voltage was adjusted to 120 V until electrophoresis was completed.

5.5) Membrane transfer: the PVDF membrane, activated by the methanol solution, was placed in a transfer solution, and the desired proteins were cut down according to molecular weight, and white transfer clips, sponges, filter paper, PVDF membranes, gel, filter paper, sponges, and white transfer clips were placed in order, and air bubbles must be avoided during the operation. Two ice boxes were placed in a wet transfer tank, and then the tank was placed on ice, and finally the pre-cooled wet transfer solution was added. The membrane was transferred for 90 min at a constant current of 220 mA.

5.6) Sealing: the PVDF membrane was soaked in 5% skimmed milk (TBST preparation), sealed at room temperature for 1 h, and washed once with TBST.

5.7) Primary antibody incubation: the cut protein bands were placed into the corresponding primary antibody and incubated at 4° C. on a shaker overnight, washed with TBST 3 times for 10 min/time.

5.8) Secondary antibody incubation: the corresponding secondary antibody was incubated at room temperature for 1 h., washed with TBST 3 times, 10 min/time.

5.9) Protein strip development: luminescent solution 1:1 was prepared at dark conditions; TBST on the protein strip was adsorbed with filter paper; the luminescent solution was applied evenly on the strip, and finally the protein strip was developed with a luminescent imager.

### 6. Tissue RNA Extraction and its Reverse Transcription

6.1) Sample preparation: 15-20 mg of mouse skin tissue was placed in a 1.5 mL centrifuge tube, and 600 μL of pre-cooled lysate was quickly added, and homogenized with an electric homogenizer. The homogenate obtained was blown gently 10 times, left at room temperature for 3-5 min, then centrifuged at 14,000×g for 5 min, and the supernatant was transferred to a new centrifuge tube with a pipette gun of suitable volume.

The amount of mouse skin tissue generally was not more than 30 mg, and excessive amounts led to decreased acquisition rates due to insufficient lysis. After lysis and centrifugation of the tissue sample, there may be some gelatinous material in the lower part of the centrifuge tube, which was suitable to be transferred as supernatant to the next operation. If the gelatinous material was discarded, this would result in a decrease in yield of approximately 30-50%. The gelatinous material would disappear with the addition of the binding solution. Centrifugation was used to remove obvious lumps that have not been homogenized sufficiently. If a sticky mass remained after lysis, additional blowing was required until the solution was completely clarified. For RNase-rich samples, it is advisable to add DTT to the lysate to a final concentration of 40 mM or β-mercaptoethanol to a final concentration of 1%.

6.2) Add an equal volume of a binding solution to the lysate and mix gently by inverting 3-5 times. Precipitate is normal.

6.3) The mixture (including precipitate) was transferred to a purification column, centrifuged at 12,000×g for 30 sec, and the liquid in the collection tube was discarded.

When the volume of lysate was larger than 300 μL, after an equal volume of binding solution was added, the total volume would exceed the capacity of the purification column. Thus, the lysate was divided into 2 parts, i.e., half of the mixture passed through the column, and then the remaining mixture was treated according to step 6.3) once again. For some special samples, if the solution failed to pass through completely, the centrifugation time can be extended to 1-2 min, or the centrifugation force can be increased to 16,000×g.

6.4) Add 600 μL of Wash Solution I, centrifuge at 12,000×g for 30 sec, and discard the liquid in the collection tube.

6.5) Add 600 μL of Wash Solution II, centrifuge at 12,000×g for 30 sec, and discard the liquid in the collection tube.

6.6) Repeat step 6.5) once.

6.7) Centrifuge at the highest speed (about 14,000-16,000×g) for 2 min to remove residual liquid.

6.8) Place the RNA purification column in the RNA elution tube provided in the Biyun Tian RNAeasy™ Animal RNA Extraction Kit (Centrifugal Column), add 30-50 μL of eluent, leave it at room temperature for 2-3 min, and centrifuge it at the highest speed for 30 sec, and the resultant solution was the purified RNA.

The eluent was added to the center of the surface of the purification column so that it was completely absorbed. When the room temperature was low, the eluent was preheated at 37° C. for a few moments. The eluted solution was added back to the original purification column and centrifuged to elute again, which could increase the yield by about 10-30%, or eluted again with a new eluent after the first elution, which would yield about 15-40% of the RNA of the first elution.

6.9) The concentration and purity of RNA were measured using the NanoDrop 2000 Nucleic Acid Protein Analyzer, and the obtained RNA concentration was diluted to 1 μg/μL with DEPC water.

6.10) Reverse transcribe the RNA into cDNA according to the instructions of the reverse transcription kit, and then carry out subsequent experiments or store at −20° C. for later use.

### 7. Real-Time Fluorescence Quantitative PCR Assay

7.1) Dilute the specific gene amplification primers to 10 UM in ddH2O, prepare the qPCR reaction system according to the instructions in the kit and add them sequentially into a 384-well plate.

The reaction system was as follows:

| Agents | Volume | |
|---|---|---|
| 2 × AceQ qPCR SYBR Green Master Mix | 5 | μL |
| ddH₂O | 3.4 | μL |
| Primer F | 0.2 | μL |
| Primer R | 0.2 | μL |
| ROX 1 | 0.2 | μL |
| cDNA | 1 | μL |
| Total voume | 10 | μL |

7.2) Seal the 384-well plate with a plate sealing film, centrifuge at 3000 rpm for 1 min at room temperature for qPCR amplification, and preheat the ABI 7900HT PCR instrument with the following thermal cycling conditions:

| Stage | Temperature | Time | | Number of cycles |
|---|---|---|---|---|
| Pre-mutagenesis | 95° C. | 5 | min | 1 |
| Cyclic reaction | 95° C. | 10 | sec | 40 |
| | 60° C. | 30 | sec | |
| | 95° C. | 15 | sec | |
| Dissolution curves | 60° C. | 1 | min | 1 |
| | 95° C. | 15 | sec | |

7.3) Relative mRNA expression levels of genes were calculated by the 2-44CT relative quantification method using GAPDH as an internal reference.

### 8. H&E Staining

Basic procedure for making HE-stained paraffin sections:

8.1) Sampling and fixation: After the experiment, skin tissue was taken from the depilated area on the back of the mouse and placed in a 10 mL EP tube. 4% paraformaldehyde was added to the EP tube, and all tissues were immersed to denature and coagulate the proteins of the skin tissue. The original morphology and structure of the cells were maintained.

8.2) Dewatering: with low to high concentrations of ethanol as a dehydrating agent, the water in the tissue blocks was gradually removed in the order of 75% ethanol for 5 minutes, 85% ethanol for 5 minutes, 95% ethanol for 5 minutes, anhydrous ethanol I for 5 minutes, and anhydrous ethanol II for 5 minutes. The tissue blocks were then placed in xylene, a transparent agent that is soluble in both ethanol and paraffin, and the ethanol in the tissue blocks was replaced with xylene.

8.3) Wax dipping and embedding: transparent skin tissue blocks were placed in melted paraffin wax and kept warm in the waxing box. The tissue block was completely immersed in the paraffin wax for embedding: specifically, the melted paraffin wax was poured in a container prepared in advance, and the paraffin-impregnated tissue block was quickly added to the container. The block was cooled and solidified.

8.4) Slicing and patching: the embedded wax block was fixed on a slicer and cut into thin slices of about 5-8 μm. The slices were wrinkled and needed to be ironed out in heated water and finally pasted onto slides and dried at 45° C. in a thermostat.

8.5) Deparaffinization and staining: H&E staining increases the difference in color between the various parts of a tissue's cellular structure for easier visualization. Hematoxylin (H) is a basic dye that stains the nucleus and intracellular ribosomes blue-violet, and structures stained with hematoxylin are basophilic. Eosin (E) is an acidic dye that stains the cytoplasm of cells red or light red, and the structures stained with eosin are eosinophilic. Before staining, the tissue sections were sequentially placed in xylene I for 30 min, xylene II for 30 min, anhydrous ethanol I for 5 min, anhydrous ethanol II for 5 min, 95% ethanol for 5 min, 85% ethanol for 5 min, 75% ethanol for 5 min and PBS for 5 min.

8.6) H&E staining: the sections pretreated in distilled water were put into aqueous hematoxylin solution for a few minutes for staining. The color was separated in acid water and ammonia for a few seconds each. After rinsing in running water for 1 h, the sections were placed in distilled water for a few moments. The sections were dehydrated in 70% and 90% alcohol for 10 min each, and stained in alcoholic eosin stain for 2-3 min.

8.7) Dewatering and transparency: after staining, the sections were gradually dewatered in anhydrous ethanol I for 5 min, anhydrous ethanol II for 5 min, and the sections were made transparent by xylene.

8.8) Sealing: the transparent section was covered with a drop of gum and covered with a coverslip. After the gum was dried slightly, label the section.

### 9. Immunohistofluorescence Staining Assay

9.1) Drying: skin tissue sections were placed in an oven at 60° C. for 2 hours.

9.2) Deparaffinization: the tissue sections were sequentially placed in xylene I for 30 min, xylene II for 30 min, anhydrous ethanol I for 5 min, anhydrous ethanol II for 5 min, 95% ethanol for 5 min, 85% ethanol for 5 min, 75% ethanol for 5 min, and PBS for 5 min.

9.3) Antigen repair: the sections were placed in citric acid antigen repair buffer for antigen repair and then washed 3 times in PBS on a shaker.

9.4) Sealing: seal with goat serum for 1 h, wash with PBS once.

9.5) Primary antibody incubation: the tissue was circled by the histochemical pen, about 50 μL of primary antibody was added dropwise to the tissue, and incubated in a wet box overnight at 4° C., washed 3 times with PBS.

9.6) Secondary antibody incubation: add about 50 μL of a corresponding fluorescent secondary antibody dropwise, in dark condition, incubate at room temperature for 2 h. Wash with PBS 3 times.

9.7) Photographing: DAPI-containing anti-fluorescence quenching sealer was added dropwise to the tissues, sealed with coverslips, and the tissues were incubated for 5 min at room temperature in dark condition; the molecular expression was observed with a Zeiss LSM900 confocal microscope, and the fluorescence intensity was quantified by Image J.

### 10. Analysis of Public Databases for Bioinformatics

10.1) The correlation between the molecules required for the study of hair loss areas and non-hair loss areas in AGA patients was queried in the GEO database (https://www.ncbi.nlm.njh.gov/geo/) and used to further support the experimental results.

10.2) Predict the transcription factor with Animal TFDB (https://guolab.wchscu.cn/AnimalTFDB4//#/), PROMO (https://alggen.lsi.upc.es/cgibin/promo_v3/promo/promoinit.cgi?dirDB=TF_8.3), JASPAR (https://jaspar.elixir.no/).

### 10.3) Proteomics and Protein Phosphomics Sequencing

Appropriate amounts of mouse skin tissues (n=3) from the model group and 1% JP1 group were added to a mortar pre-cooled with liquid nitrogen, and ground thoroughly with liquid nitrogen into powders. The samples were separately added to lysis buffer and lysed by ultrasonication. The centrifugation was performed at 4° C., 12,000×g for 10 min, and protein concentration was determined by BCA. Trypsin digestion was followed by liquid chromatography-mass spectrometry serial analysis. The identified proteins were analyzed by KEGG enrichment, and Log 2 Fold enrichment >1 was defined as significantly up-regulated.

### 11. Statistic Analysis

Measurement data in the study were expressed as mean±standard deviation. The test of difference between two groups was analyzed by Student's t-test, the statistical analysis of quantitative data of multiple groups was analyzed by ANOVA (One-way analysis of variance), and the correlation analysis adopted Pearson correlation analysis. *P<0.05, **P<0.01, ***P<0.001 indicated statistically significant differences, and ns P>0.05 indicated no statistically significant differences between groups. Analysis and graphing utilized PSS 20.0 and GraphPad Prism 8.0.1.

It will be obvious to those skilled in the art that changes and modifications may be made, and therefore, the aim in the appended claims is to cover all such changes and modifications.

What is claimed is:

1. A method for treating or preventing alopecia comprising administering a patient in need thereof a pharmaceutical composition comprising a polypeptide, the peptide having an amino acid sequence I or II:
I: FPGSDRF (SEQ ID NO: 1)-Z;
II: X-FPGSDRF (SEQ ID NO: 1)-Z;
wherein:
S represents phosphorylated serine;
X and Z independently represent an amino acid or an amino acid sequence;
X is selected from F, (R)₉ (SEQ ID NO: 2), (R)₉-F (SEQ ID NO: 3), 6-aminohexanoic acid, 6-aminohexanoic acid-F, 6-aminohexanoic acid-(R)₉, 6-aminohexanoic acid-(R)₉-F; and
Z is selected from (G)ₙ-RGD or A-(G)ₙ-RGD (SEQ ID NO: 4), where n is an integer greater than or equal to 0, in the range of 0-10.

2. The method of claim 1, wherein the alopecia is androgenetic alopecia.

3. The method of claim 1, wherein the pharmaceutical composition is configured to target hair follicle tissues, promote a growth of hair follicles and hair rods, shorten a resting period of hair follicles, prolong a growth period of hair follicles, and significantly reverse an inhibition of hair growth by androgens.

4. The method of claim 1, wherein the pharmaceutical composition is configured to target integrin molecules into hair follicle cells and upregulate an expression of integrin molecules in a hair follicle cell membrane; and the integrin molecules comprise αvβ1.

5. The method of claim 1, wherein the pharmaceutical composition is configured to promote an expression of a nuclear transcription factor SP1 by activating a MEK/ERK/E2F1 signaling axis, and the nuclear transcription factor SP1 in turn activates a Wnt/β-catenin signaling pathway in hair follicle stem cells.

6. The method of claim 1, wherein the polypeptide comprises an acetylated N-terminal and an amidated C-terminal.

7. The method of claim 1, wherein an amino acid sequence of the polypeptide is one of SEQ ID NO: 5 to SEQ ID NO: 27.

8. The method of claim 1, wherein each amino acid in the FPGSDRF sequence is either L-type or D-type.

9. The method of claim 1, wherein the pharmaceutical composition further comprises a pharmaceutically acceptable excipient.

10. The method of claim 1, wherein a dosage form of the pharmaceutical composition is injectable or for external use.
